SEQUENCE LISTING (5 sequences)

SEQ ID NO: 1 - DNA, 3645 bp, Homo sapiens
cctgaagggaggatgggctaaggcaggcacacagtggcggagaagatgccctcctgggccctcttcatggtcacctcctgcctcctcctggcccctcaaaacctggcccaagtcagcagccaagatgtctccttgctggcatcagactcagagcccctgaagtgtttctcccgaacatttgaggacctcacttgcttctgggatgaggaagaggcagcgcccagtgggacataccagctgctgtatgcctacccgcgggagaagccccgtgcttgccccctgagttcccagagcatgccccactttggaacccgatacgtgtgccagtttccagaccaggaggaagtgcgtctcttctttccgctgcacctctgggtgaagaatgtgttcctaaaccagactcggactcagcgagtcctctttgtggacagtgtaggcctgccggctccccccagtatcatcaaggccatgggtgggagccagccaggggaacttcagatcagctgggaggagccagctccagaaatcagtgatttcctgaggtacgaactccgctatggccccagagatcccaagaactccactggtcccacggtcatacagctgattgccacagaaacctgctgccctgctctgcagaggcctcactcagcctctgctctggaccagtctccatgtgctcagcccacaatgccctggcaagatggaccaaagcagacctccccaagtagagaagcttcagctctgacagcagagggtggaagctgcctcatctcaggactccagcctggcaactcctactggctgcagctgcgcagcgaacctgatgggatctccctcggtggctcctggggatcctggtccctccctgtgactgtggacctgcctggagatgcagtggcacttggactgcaatgctttaccttggacctgaagaatgttacctgtcaatggcagcaacaggaccatgctagctcccaaggcttcttctaccacagcagggcacggtgctgccccagagacaggtaccccatctgggagaactgcgaagaggaagagaaaacaaatccaggactacagaccccacagttctctcgctgccacttcaagtcacgaaatgacagcattattcacatccttgtggaggtgaccacagccccgggtactgttcacagctacctgggctcccctttctggatccaccaggctgtgcgcctccccaccccaaacttgcactggagggagatctccagtgggcatctggaattggagtggcagcacccatcgtcctgggcagcccaagagacctgttatcaactccgatacacaggagaaggccatcaggactggaaggtgctggagccgcctctcggggcccgaggagggaccctggagctgcgcccgcgatctcgctaccgtttacagctgcgcgccaggctcaacggccccacctaccaaggtccctggagctcgtggtcggacccaactagggtggagaccgccaccgagaccgcctggatctccttggtgaccgctctgcatctagtgctgggcctcagcgccgtcctgggcctgctgctgctgaggtggcagtttcctgcacactacaggagactgaggcatgccctgtggccctcacttccagacctgcaccgggtcctaggccagtaccttagggacactgcagccctgagcccgcccaaggccacagtctcagatacctgtgaagaagtggaacccagcctccttgaaatcctccccaagtcctcagagaggactcctttgcccctgtgttcctcccaggcccagatggactaccgaagattgcagccttcttgcctggggaccatgcccctgtctgtgtgcccacccatggctgagtcagggtcctgctgtaccacccacattgccaaccattcctacctaccactaagctattggcagcagccttgaggacaggctcctcactcccagttccctggacagagctaaactctcgagacttctctgtgaacttccctaccctacccccacaacacaagcaccccagacctcacctccatccccctctgtctgccctcacaattaggcttcattgcactgatcttactctactgctgctgacataaaaccaggaccctttctccacaggcaggctcatttcactaagctcctcctttactttctctctcctctttgatgtcaaacgccttgaaaacaagcctccacttccccacacttcccatttactcttgagactacttcaattagttcccctactacactttgctagtgaaactgcccaggcaaagtgcacctcaaatcttctaattccaagatccaataggatctcgttaatcatcagttcctttgatctcgctgtaagatttgtcaaggctgactactcacttctcctttaaattctttcctaccttggtcctgcctctttgagtatattagtaggttttttttatttgtttgagacagggtctcactctgtcacccaggctgcagtgcaatggcgcgatctcagctcactgcaacctccacctccgggttcaagcgattcttgtgcctcggcctccctagtagctgggattacaggcgcacaccaccacacacagctaattttttttttttttttttttttttttttttagacggagccttgctctgttgccagactggagtgcagtggcacgatctcggctcactgcaacctctgcctcccgggttcaagccattctgcctcagcctcccaagtagctgggagtacaggcgtctgccaccatgcctaatttttttctatttttaggagagaccggttttcaccacgttggccaggatggtctcgatatcctgatctcgtgatccgcctgcctctgcctcccaaagtgctgggattacaggtgtgacccactgcgcacagccccagctaattttcatatttttagtagagacagggttttgccatgttgcccaggctggtcttgaactcctaacctcgggtgatccacccaccttggcctcccaaagtgttaggattacaggcatgagccactgcgcccggctgagtgtactagtagttaagagaataaactagatctagaatcagagctggattcaattcctgtccttcacatttactagctgtgcaaccttgggcacataacttaatgtctttgagccttagttttttcatctgtaaaacagggataataacagcaccccatagagttgtgacgaggattgagataatctaagtaaagcacagtccctaggacatagtaaatgattcatatatccgaactactgttataattattccttcttactctcctcttctagcatttcttccaattattacagtccttcaagattccatttcttaacagtctccaatcccatctattctctgcctttactatatgttgaccattccaaagttcttatctctagctcagacatctactacagcactgtgatgctttatgcaactaactgtttacatatctgtcccctgctactagattgtgagctccttgagggaaaggaacatgatttatttgtccttttcccccagcacctagagtagtgcttggtgcatgatagtaggccttcaataaattttttctaaatgaatga

SEQ ID NO: 2 - protein, 635 aa, Homo sapiens
MPSWALFMVTSCLLLAPQNLAQVSSQDVSLLASDSEPLKCFSRTFEDLTCFWDEEEAAPSGTYQLLYAYPREKPRACPLSSQSMPHFGTRYVCQFPDQEEVRLFFPLHLWVKNVFLNQTRTQRVLFVDSVGLPAPPSIIKAMGGSQPGELQISWEEPAPEISDFLRYELRYGPRDPKNSTGPTVIQLIATETCCPALQRPHSASALDQSPCAQPTMPWQDGPKQTSPSREASALTAEGGSCLISGLQPGNSYWLQLRSEPDGISLGGSWGSWSLPVTVDLPGDAVALGLQCFTLDLKNVTCQWQQQDHASSQGFFYHSRARCCPRDRYPIWENCEEEEKTNPGLQTPQFSRCHFKSRNDSIIHILVEVTTAPGTVHSYLGSPFWIHQAVRLPTPNLHWREISSGHLELEWQHPSSWAAQETCYQLRYTGEGHQDWKVLEPPLGARGGTLELRPRSRYRLQLRARLNGPTYQGPWSSWSDPTRVETATETAWISLVTALHLVLGLSAVLGLLLLRWQFPAHYRRLRHALWPSLPDLHRVLGQYLRDTAALSPPKATVSDTCEEVEPSLLEILPKSSERTPLPLCSSQAQMDYRRLQPSCLGTMPLSVCPPMAESGSCCTTHIANHSYLPLSYWQQP

SEQ ID NO: 3 - protein, 19 aa, Artificial Sequence
  Synthetic primer
GTCCAGCCAGTGTACTAAT

SEQ ID NO: 4 - protein, 17 aa, Artificial Sequence
  Synthetic primer
CTCAGGCCGAATTCCAT

SEQ ID NO: 5 - protein, 21 aa, Artificial Sequence
  Synthetic primer
CTGGAGATGTTGAGAGCAATC

(19) United States
(12) Patent Application Publication
(10) Pub. No.: US 2020/0016207 A1
(43) Pub. Date: Jan. 16, 2020

(54) TRANSGENIC c-MPL PROVIDES LIGAND-DEPENDENT CO-STIMULATION AND CYTOKINE SIGNALS TO TCR-ENGINEERED T CELLS

(71) Applicant: Baylor College of Medicine, Houston, TX (US)

(72) Inventors: Caroline Eva Arber Barth, Bellaire, TX (US); Malcolm K. Brenner, Bellaire, TX (US)

(21) Appl. No.: 16/495,184

(22) PCT Filed: Mar. 20, 2018
(86) PCT No.: PCT/US2018/023408

§ 371 Date: Sep. 18, 2019

Related U.S. Application Data
(60) Provisional application No. 62/473,679, filed on Mar. 20, 2017.

(51) Int. Cl. A61K 35/17; C12N 5/0783; C07K 14/725; C07K 14/715; A61P 35/00; A61P 35/02; A61K 45/06; A61K 38/17

(52) U.S. Cl. CPC A61K 35/17; C12N 5/0636; C07K 14/7051; C07K 14/715; A61P 35/00; C12N 2501/20; A61K 45/06; A61K 38/1793; A61K 38/1774; C12N 2510/00; A61P 35/02

(57) ABSTRACT

Embodiments of the present disclosure concern improvements to cell therapy for cancer. In certain embodiments, an immune cell lacks expression of hematopoietic growth factor receptor c-MPL (myeloproliferative leukemia), the receptor for thrombopoietin (TPO), and supplementation of this effect allows an improvement for cancer cell therapy, including of hematological malignancies. In specific embodiments, immune cells comprise recombinant c-MPL expression or parts thereof and the cells have enhanced co-stimulation and cytokine signals and improved activation, persistence, and anti-tumor function compared to cells that lack recombinant c-MPL expression.

This application claims priority to U.S. Provisional Patent Application Ser. No. 62/473,679, filed Mar. 20, 2017, which is incorporated by reference herein in its entirety.

### STATEMENT REGARDING FEDERALLY SPONSORED RESEARCH OR DEVELOPMENT

This invention was made with government support under P50CA126752 awarded by NIH-NCI SPORE and P30CA125123 awarded by the NIH. The government has certain rights in the invention.

### TECHNICAL FIELD

Embodiments of the present disclosure concern at least the fields of cell therapy, immunotherapy, molecular biology, cell biology, and medicine, including cancer medicine.

### BACKGROUND OF THE INVENTION

T cells modified with a transgenic T cell receptor (TCR) can efficiently target intracellular tumor-associated antigens (TAAs) processed and presented on the cell surface in the context of major histocompatibility complex (MHC) molecules (Kershaw et al., 2013; Fesnak et al., 2016). These TAAs include lineage differentiation antigens, cancer testis antigens and the inhibitor of apoptosis protein, surviving (Cheever et al., 2009). While transgenic TCRs mediate specific target antigen recognition (signal 1), TCR-transgenic T cells lack built-in transgenic co-stimulation (signal 2) to enhance antigen-specific responses, a distinction from “second generation” chimeric antigen receptor (CAR)-modified T cells (Fesnak et al., 2016; Dotti et al., 2014). Most engineered T cells of both types rely on host-derived cytokine signals (signal 3) for their sustained in vivo function and persistence, but levels vary in individual patients. In addition, cytokines may not efficiently reach the tumor site where they are most needed for the support of adoptively transferred T cells. A cytokine milieu more favorable to expansion and effector function can be induced by administration of lymphodepleting chemotherapy to the patient prior to adoptive T cell therapy, but may be insufficiently sustained for optimal anti-tumor activity. It was therefore investigated whether a single additional gene modification incorporating both signals 2 and 3 would more consistently and controllably improve TCR-transgenic T cell persistence and anti-tumor function in vivo, with a receptor that responds both to a tumor microenvironment factor and to pharmacological agents.

The hematopoietic growth factor receptor c-MPL (myeloproliferative leukemia) is the receptor for thrombopoietin (TPO) and is expressed in hematopoietic stem cells (HSCs) and progenitor cells of the myelo/megakaryocytic lineage (Hitchcock and Kaushansky, 2014). C-MPL is involved in self-renewal, expansion and maintenance of the HSC pool, stimulation of megakaryocytic progenitor cells supporting platelet production and maturation, but is not expressed in lymphoid lineage cells (Kaushansky et al., 1994; Fox et al., 002; Qian et al., 2007). TPO is produced in the liver, kidneys and in the bone marrow (BM) microenvironment by stem-cell niche cells where it locally supports HSCs and progenitors (Yoshihara et al., 2007; Schepers et al., 2013); its systemic levels are tightly regulated by c-MPL-mediated TPO scavenging (Chang et al., 1996) as well as sensing of aged platelets by the Ashwell-Morell receptor in the liver (Grozovsky et al. 2015). TPO binding to c-MPL activates several signaling pathways including JAK2/STAT, PI3K/Akt, and Raf-1/MAP kinase, in addition to activation of its negative regulator SOCS-3 (Hitchcock and Kaushansky, 2014). Thus, c-MPL activated pathways significantly overlap with common pathways used by T cell co-stimulatory molecules (e.g. CD28) (Chen and Flies, 2013) as well as common γ-chain cytokine receptors (e.g. IL-2, 4, 7, 9, 15, 21) (Rochman et al., 2009), so that human T cells engineered to express a transgenic c-MPL receptor should receive both co-stimulatory (signal 2) and cytokine signals (signal 3) upon c-MPL activation. We therefore determined (a) whether systemic TPO levels in steady-state could provide homeostatic expansion signals to c-MPL-transgenic T cells, (b) if local BM microenvironment TPO levels were sufficient to support local anti-tumor function and persistence of TAA-specific TCR-transgenic c-MPL+ T cells that targeted hematologic malignancies, and (c) whether pharmacologic support of c-MPL+ TCR-transgenic T cells could further enhance their anti-tumor activity.

It is shown herein that c-MPL can be efficiently expressed in polyclonal as well as tumor-targeted TCR-transgenic T cells. C-MPL activation of T cells under steady-state conditions increases T cell persistence, and enhances the anti-tumor activity of TCR-transgenic T cells in vitro and in vivo. In addition to increased T cell expansion and persistence, c-MPL activation of transgenic T cells increased their cytokine production, and led to more efficient immune synapse formation; these effects were associated with significant changes in gene expression signatures affecting pro-inflammatory and cell cycle pathways. Hence, c-MPL can mediate both co-stimulation and cytokine signals (2 and 3) in T cells and thereby improve their anti-tumor activities.

The present disclosure provides solutions to a long-felt need in the art for enhanced adoptive cell therapy.

### BRIEF SUMMARY OF THE INVENTION

The present disclosure is directed to compositions and methods related to cell therapy. In particular embodiments, the cell therapy is for an individual in need of cell therapy, such as a mammal, including a human, dog, cat, horse, etc. The cell therapy may be suitable for any medical condition, although in specific embodiments the cell therapy is for cancer. The cancer may be of any kind, although in specific embodiments the cancer comprises one or more hematological malignancies, such as leukemia or lymphoma. The individual may be of any age or gender. In specific embodiments, the individual is known to have cancer or may be suspected of having cancer or be at risk for cancer. The cancer may be a primary or metastatic cancer, and the cancer may or may not be refractory to treatment. In some embodiments, the cancer concerns treatment of solid tumors, such as tumors of the brain, breast, bladder, colon, rectum, kidney, liver, lung, ovary, pancreas, prostate, and so forth, for example. In specific embodiments, the disclosure concerns treatment of non-solid tumors, such as acute lymphoblastic leukemia, acute myelogenous leukemia, chronic lymphocytic leukemia, chronic myelogenous leukemia, acute monocytic leukemia, Hodgkin's lymphoma, non-Hodgkin's lymphoma, and so forth, for example.

In one embodiment, there is an immune cell or population thereof comprising recombinant expression of the thrombopoietin receptor (c-MPL). In specific cases, there is no expression of endogenous c-MPL in the cell and in other specific cases there is an existing expression of c-MPL that is overexpressed upon recombinant expression of c-MPL. The immune cell may be an alpha beta T cell, gamma delta T cell, NK cell, or NKT cell, tumor infiltrating lymphocyte, or marrow infiltrating lymphocyte, for example. The immune cell may or may not comprise an engineered receptor, such as a transgenic T cell receptor (TCR) or a chimeric antigen receptor (CAR). In specific cases, the engineered receptor targets a tumor-associated antigen, such as EphA2, HER2, GD2, Glypican-3, 5T4, 8H9, αvβ6 integrin, B cell maturation antigen (BCMA) B7-H3, B7-H6, CAIX, CA9, CD19, CD20, CD22, kappa light chain, CD30, CD33, CD38, CD44, CD44v6, CD44v7/8, CD70, CD123, CD138, CD171, CS1, CSPG4, EGFR, EGFRvIII, EGP2, EGP40, EPCAM, ERBB3, ERBB4, ErbB3/4, FAP, FAR, FBP, fetal AchR, Folate Receptor α, GD2, GD3, HLA-AI, HLA-A2, IL11Ra, IL13Ra2, KDR, Lambda, Lewis-Y, MCSP, Mesothelin, Muc1, Muc16, NCAM, NKG2D ligands, NY-ESO-1, PRAME, PSCA, PSC1, PSMA, ROR1, Sp17, SURVIVIN, TAG72, TEM1, TEM8, carcinoembryonic antigen, HMW-MAA, VEGF receptors, MAGE-A1, MAGE-A3, MAGE-A4, CT83, SSX2, XIAP, cIAP1, cIAP2, NAIP, and/or Livin.

In specific cases for the cell, c-MPL is expressed via a recombinant expression vector operable in eukaryotic cells, and the expression of c-MPL may be regulated by a constitutive promoter or an inducible promoter. In specific embodiments, the vector is a viral vector, such as a retrovirus, lentivirus, adenovirus, adeno-associated virus, or herpes simplex virus, or the vector is a non-viral vector, such as naked DNA or plasmid DNA or minicircle DNA. In particular cases, the c-MPL is a functionally active fragment or variant of c-MPL.

In one embodiment, there is a method of improving immune cell therapy, comprising the step of modifying the immune cells to express c-MPL or functional parts thereof. In specific cases, the cells comprise the cells of the disclosure. The cell therapy may be for a malignancy in an individual, such as one that comprises acute lymphoblastic leukemia, acute myelogenous leukemia, chronic lymphocytic leukemia, chronic myelogenous leukemia, acute monocytic leukemia, Hodgkin's lymphoma, non-Hodgkin's lymphoma, and/or solid tumors. In specific cases, the solid tumors comprise tumors of the brain, breast, bladder, bone, colon, rectum, cervix, endometrium, esophagus, eye, gallbladder, hypopharynx, kidney, larynx, liver, lung, nasopharynx, oropharynx, ovary, pancreas, penis, pituitary, prostate, skin, small intestine, stomach, testes, thymus, thyroid, uterus, vagina and/or vulva.

In a particular embodiment, there is a method for improving immune cell persistence and/or function, comprising the step of activating the immune cells that express recombinant c-MPL by subjecting the cells to thrombopoietin (TPO) and/or one or more agonists of c-MPL. Any of the cells encompassed herein may be utilized in any method. In specific cases, the activating step occurs ex vivo, in vitro, or in vivo. In at least some cases, the cells are exposed to TPO. The cells may be exposed to one or more agonists of c-MPL, such as eltrombopag (EP), NIP-004 or other small molecule agonists, romiplostim or other peptide agonists, or a combination thereof.

In one embodiment, there is a method for treating cancer in an individual, comprising the step of delivering to the individual a therapeutically effective amount of immune cells of the disclosure. In specific embodiments, the method further comprises the step of exposing immune cells of the disclosure to TPO and/or one or more agonists of c-MPL. In specific embodiments, the cancer comprises acute lymphoblastic leukemia, acute myelogenous leukemia, chronic lymphocytic leukemia, chronic myelogenous leukemia, acute monocytic leukemia, Hodgkin's lymphoma, non-Hodgkin's lymphoma, and/or solid tumors, such as tumors of the brain, breast, bladder, bone, colon, rectum, cervix, endometrium, esophagus, eye, gallbladder, kidney, larynx and hypopharynx, liver, lung, nasopharynx, oropharynx, ovary, pancreas, penis, pituitary, prostate, skin, small intestine, stomach, testes, thymus, thyroid, uterus, vagina and/or vulva. The individual may be provided one or more additional cancer therapies, such as chemotherapy, radiation, immunotherapy, surgery, or a combination thereof.

As demonstrated herein, (a) systemic TPO levels in steady-state can provide homeostatic expansion signals to c-MPL-transgenic T cells, (b) local BM microenvironment TPO levels are sufficient to support local anti-tumor function and persistence of TAA-specific TCR-transgenic c-MPL+ T cells that targeted hematologic malignancies, and (c) pharmacologic support of c-MPL+ TCR-transgenic T cells enhances their anti-tumor activity.

The inventors demonstrate that c-MPL can be efficiently expressed in polyclonal as well as tumor-targeted TCR-transgenic T cells. C-MPL activation of T cells under steady-state conditions increases T cell persistence, and enhances the anti-tumor activity of TCR-transgenic T cells in vitro and in vivo. In addition to increased T cell expansion and persistence, c-MPL activation of transgenic T cells increased their cytokine production, and led to more efficient immune synapse formation; these effects were associated with significant changes in gene expression signatures affecting pro-inflammatory and cell cycle pathways. Hence, c-MPL can mediate both co-stimulation and cytokine signals (2 and 3) in T cells and thereby improve their anti-tumor activities.

The foregoing has outlined rather broadly the features and technical advantages of the present invention in order that the detailed description of the invention that follows may be better understood. Additional features and advantages of the invention will be described hereinafter which form the subject of the claims of the invention. It should be appreciated by those skilled in the art that the conception and specific embodiment disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims. The novel features which are believed to be characteristic of the invention, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present invention.

### BRIEF DESCRIPTION OF THE DRAWINGS

For a more complete understanding of the present invention, reference is now made to the following descriptions taken in conjunction with the accompanying drawing, in which:

FIGS. 1A-1H: C-MPL expression in polyclonal human T cells produces agonist-dependent proliferation and increased persistence in vivo. (FIG. 1A) c-MPL expression in polyclonal CD4+ and CD8+ T cells 7 days after retroviral transduction, representative FACS plots (left) and summary (right, n=5). Non-transduced cells (NT) black circles, c-MPL-transduced (c-MPL+) red squares, mean±SD. (FIG. 1B) Expansion of NT (left) or c-MPL+ (right) T cells cultured in no cytokines (no CK, black circles, solid lines), TPO 50 ng/ml (red squares, solid lines), or IL-2 50 U/ml (black triangles, dashed lines) for 7 days. Replicates for n=4 donors. (FIG. 1C) CFSE dilution of c-MPL-transduced cells cultured in no CK (black), TPO50 ng/ml (red) or IL-2 50 U/ml (grey) for 7 days, gated on c-MPL− (left) or c-MPL+ (right) cells. 1 representative donor of 3. (FIG. 1D) c-MPL ligand induced STATS phosphorylation in c-MPL+ T cells after treatment for 1 or 24 hours with no cytokines (black), TPO 5 ng/ml (red), TPO 50 ng/ml (blue) or eltrombopag (EP 0.1 μg/ml, green). (FIG. 1E) Mouse model experimental set up. (FIG. 1F) Transduction efficiency of T cells transduced with GFP-ffLuc alone (top panel) or co-transduced with GFP-ffLuc and c-MPL (lower panel) and injected i.v. into unconditioned hTPOtg-RAG2^{-/-}γc^{-/-} mice. (FIG. 1G) Summary of bioluminescent imaging data of control T cells (GFP-ffLuc+, black circles and lines, n=10) or c-MPL+ T cells (GFP-ffLuc+c-MPL+, red squares and lines, n=8). *p=0.0003, GFP-ffLuc+vs GFP-ffLuc+c-MPL+, t-test on log area under the curve (AUC) for second T cell infusion. Combined results from 2 independent experiments. (FIG. 1H) 3 representative mice/group imaged over time by BLI, color scale 5×10³ to 5×10⁴ p/sec/cm²/sr.

FIGS. 2A-2F: C-MPL is functional in survivin-specific TCR-transgenic T cells and enhances anti-tumor function in vitro. (FIG. 2A) Tansduction efficiencies of CD8+ activated T cells with survivin-TCR alone (murine constant β chain, mCβ) or in combination with c-MPL. Representative FACS plots (left) and summary (right), n=13, mean±SD. (FIG. 2B) TCR+c-MPL+ T cells expand upon stimulation with survivin peptide pulsed artificial antigen presenting cells (aAPCs) in a TPO dose responsive manner (right), TCR+ T cells only expand in IL-2 but not high dose TPO (left), n=6, except for no CK condition (n=3), mean±SD. TCR+ T cells at end S2: no CK vs IL-2, p=0.003; no CK vs TPO500, p=NS. TCR+c-MPL+ T cells at end S2: no CK vs IL-2, p<0.001; no CK vs TPO5, p=NS; no CK vs TPO50, p=0.02; no CK vs TPO500, p<0.001; IL-2 vs TPO500, p=NS. t-test. (FIG. 2C) c-MPL+ T cells expand in eltrombopag in a dose-responsive manner during activation with OKT3 and CD28 antibodies, NT T cells only expand in IL-2 50 U/ml, analyzed on day 7. 1 representative of 3 donors. (FIG. 2D) c-MPL ligand (TPO or EP) induced phosphorylation of STAT3 and STATS at 1 hour (left) and 24 hours (right). (FIG. 2E) Co-culture of expanded NT, TCR+ or TCR+c-MPL+ T cells with U266 myeloma cells (HLA-A*0201+survivin+) in no cytokines (no CK, black circles), TPO 5 ng/ml (red squares), TPO 50 ng/ml (blue triangles), or IL2 25 U/ml (purple squares), effector:target ratio E:T 1:1. Residual U266 cells (left) and T cells (right) were quantified by FACS on day 5. n=3, mean±SD. (FIG. 2F) Co-culture with BV173 leukemia cells (HLA-A*0201+survivin+), E:T 1:3. Residual BV173 cells (left) and T cells (right) were quantified by FACS on day 5. n=7 for no CK, TPO5 and TPO50, n=3 for IL2, mean±SD. (FIGS. 2E, 2F) *p<0.05, **p<0.01, ***p<0.001, t-test on log transformed data. NS: not significant.

FIGS. 3A-3D: Ligand-induced c-MPL activation supports sequential killing activity and T cell expansion in TCR-transgenic T cells. (FIG. 3A) Serial co-culture with BV173 leukemia cells, E:T 1:5. Residual BV173 cells (left graph) and T cells (right graph) were quantified by FACS every 3-4 days from a total of 8 replicate wells per donor and BV173 cells were added-back to untouched wells (+) at each time-point. Cultures in no cytokine (No CK, black circles), IL2 25 U/ml (purple squares), TPO 5 ng/ml (red squares), TPO 50 ng/ml (blue trianges), EP 0.1 μg/ml (green triangles), plate-bound CD28 (black triangles, dotted line), IL2 (25 U/ml)+plate-bound CD28 (purple diamond, dotted line). n=3 for IL2, CD28, IL2+CD8, TPO5, EP; n=6 for noCK, TPO50. Lines of individual donors are shown for tumor cell counts, mean±SD for T cell counts. Left panel: serial killing activity was analyzed by Kaplan-Meier analysis, overall p<0.0001; noCK vs TPO5 p=0.007, noCK vs TPO50 p<0.0001, noCK vs EP p=0.003, noCK vs IL2 p<0.0001, noCK vs CD28 p=0.038, no CK vs CD28+IL2 p<0.0001. TPO50 vs IL2 p=NS, TPO50 vs CD28 p=0.003, TPO50 vs CD28+IL2 p=NS. Right panel: T cell expansion in no CK vs TPO5 p=NS, no CK vs TPO50 p=0.003, no CK vs EP p=NS, no CK vs IL2 p=0.001, no CK vs CD28 p=NS, no CK vs CD28+IL2 p=0.001. TPO5 vs TPO50 p=0.02, TPO5 vs EP p=NS, TPO50 vs EP p=0.03, TPO50 vs IL2 p=NS, TPO50 vs CD28+IL2 p=NS. t-test on log AUC (FIG. 3B) Cytokine levels in co-culture supernatants 24 hours after tumor cell challenge for the 1^{st}, 3^{rd}, 5^{th} and 7^{th} tumor challenge on days 1, 8, 15 and 22 of co-culture, respectively. n=3, mean±SD, analyzed in duplicates. T-test on log transformed data (days 1, 8), one sample t-test compares to null hypothesis of 0 on log transformed data (days 15, 22). *p<0.05, **p<0.01. (FIGS. 3C, 3D) T cell phenotype of CD3+CD8+TCR+c-MPL+ T cells recovered from co-cultures at each time-point, n=3-6 (as in panel A), mean±SD. (C) Percentages of CD45RA+CD45RO+ cells. No CK vs TPO5 p=0.002, noCK vs TPO50 p<0.0001, no CK vs EP p=0.002, no CK vs IL2 p=0.002, noCK vs CD28 p=NS, noCK vs CD28+IL2 p=0.004. TPO50 vs IL2 p=0.05, TPO50 vs CD28 p<0.0001, TPO50 vs CD28+IL2 p=0.005. t-test on log AUC (FIG. 3D) Naïve, central memory (CM), effector memory (EM) and effector T cells. Naïve: p=NS, except noCK vs TPO50 p=0.05, noCK vs CD28+IL2 p=0.05; CM: noCK vs TPO5 p=0.003, noCK vs TPO50 p<0.0001, noCK vs EP p=0.002, noCK vs IL2 p=0.005, noCK vs CD28 p=NS, noCK vs CD28+IL2 p=0.009. TPO50 vs IL2 p=NS, TPO50 vs CD28 p=0.001, TPO50 vs CD28+IL2 p=0.03. EP vs IL2+CD28 p=0.04. t-test on log AUC day 14.

FIGS. 4A-4C: C-MPL stimulated sequential killer T cells form more efficient immune synapses. (FIG. 4A) Experimental set up. (FIG. 4B) Representative images of immune synapses between T cells and BV173 leukemia cells. Phase contrast (left) and confocal images (right) at baseline and after co-culture. Actin (white), pericentrin (blue), perforin (green). (FIG. 4C) Quantification of the % actin at the synapse, the distance of the microtubule organization center (MTOC) to the synapse and the perforin distance to the synapse. n=3, mean±SD, **p≤0.01, t-test on log transformed data. NS: not significant.

FIGS. 5A-5E: C-MPL signaling in tumor-targeted TCR-transgenic T cells is immune stimulatory. (FIG. 5A) Heatmap of median normalized differential gene expression clustered by overall expression behavior. (FIG. 5B) Control signal mean normalized expression behavior of highlighted clusters from heatmap. (FIG. 5C) GSEA output for Reactome Interferon Alpha Beta Signaling gene set showing correlation between control and EP treatment, and control and TPO treatment. (FIG. 5D) Heatmap of enriched genes in Interferon Alpha Beta Signaling Genes. (FIG. 5E) Genes in the overlap of the Control vs EP and Control vs TPO differential genes.

FIGS. 6A-6D. C-MPL signaling in T cells significantly enhances anti-tumor function in a leukemia xenograft model. (FIG. 6A) Experimental set up. (FIG. 6B) Kaplan-Meier survival analysis. Survival of hTPOtg-RAG2^{-/-}γc^{-/-} mice, injected with BV173-ffLuc+ cells and treated with control T cells (n=7), TCR+ T cells (n=9), TCR+c-MPL+ T cells and PBS injections (n=14), TCR+c-MPL+ T cells and rhTPO injections (n=10). Results combined from 3 independent experiments. Overall survival p=0.004. TCR vs TCR+c-MPL+(PBS injected): p=0.27, TCR vs TCR+c-MPL+(rhTPO injected): p=0.001, TCR+c-MPL+(PBS injected) vs TCR+c-MPL+(rhTPO injected): p=0.07. (FIG. 6C) 3 representative mice/group imaged over time by BLI, color scale 5×10³ to 5×10⁴ p/sec/cm²/sr. (FIG. 6D) Summary of BLI data by treatment condition, results combined from 3 independent experiments, mean±SD. Control T cells (n=7, black circles solid lines), TCR+ T cells (n=9, red squares solid lines), TCR+c-MPL+ T cells (PBS injected) (n=14, blue triangles up solid lines), TCR+c-MPL+ T cells (rhTPO injected) (n=10, blue triangles down dashed lines). TCR vs TCR+c-MPL+(PBS injected): p=0.004, TCR vs TCR+c-MPL+(rhTPO injected): p=0.0005, TCR+c-MPL+(PBS injected) vs TCR+c-MPL+(rhTPO injected): p=0.088. Statistics was performed using the t-test on log AUC at week 4 compared to week 1.

FIGS. 7A-7D: Retroviral vector schemes and viral copy numbers per cell. Schematic view of (FIG. 7A) retrovirus coding for the c-MPL receptor, (FIG. 7B) retrovirus coding for the survivin-specific TCR as previously described and (FIG. 7C) retrovirus coding for both the survivin-specific TCR and c-MPL in a single vector linking the genes by 2A sequences. (FIG. 7D) Viral copy numbers per cell determined by quantitative real-time PCR.

FIG. 8. Neither c-MPL transgenic polyclonal nor TCR-transgenic T cells exhibit growth factor-independent T cell growth. T cell expansion in media alone without addition of exogenous cytokines (No CK, black circles), IL2 25 U/ml (purple squares) or TPO 50 ng/ml (blue triangles) starting 10 days after polyclonal activation with OKT3/CD28 antibodies and retroviral transduction. Non-transduced T cells (NT), TCR-transduced T cells (TCR+), c-MPL transduced T cells (C-MPL+) and TCR+c-MPL+ double transduced T cells are shown. Only T cells cultured in IL2 or c-MPL transgenic T cells cultured in TPO survived beyond day 10 of culture. Mean±SD, n=3 donors.

FIGS. 9A9B: Increased persistence of c-MPL+ T cells in mice under steady-state conditions. (FIG. 9A) Representative FACS plots of peripheral blood of mice. (FIG. 9B) Summary of FACS analysis of peripheral blood of mice on day 15-17 after T cell infusion. Detection of human T cells in peripheral blood by staining for hCD45. *p=0.04′7, t-test.

FIG. 10. TCR+c-MPL+ T cells do not exhibit growth factor-independent growth after multiple tumor cell challenges in the presence of TPO. TCR+c-MPL+ T cells were challenged three times with BV173 cells at an E:T ratio of 1:5. Continued T cell expansion was then assessed in media alone without addition of exogenous cytokines (No CK, black circles), with IL2 25 U/ml (purple squares) or TPO 50 ng/ml (blue triangles). After antigen and growth-factor withdrawal, T cells rapidly died by day 10 and did not show any signs of growth-factor independent growth. Only T cells cultured in IL2 or TPO were able to survive beyond day 10. Mean±SD, n=3 donors.

FIGS. 11A-11C: Differential expression of cell cycle genes in EP versus TPO treated sequential killer T cells. (FIG. 11A) GSEA output for Chang Cycling Genes correlation between EP treatment and TPO treatment. (FIG. 11B) Heatmap of enriched genes included in the Chang cycling genes gene set. (FIG. 11C) Normalized enrichment score versus false discovery rate q-value from GSEA analysis with cell cycle, cell growth, and proliferation signatures highlighted in red.

FIGS. 12A-12B. C-MPL stimulation supports T cell persistence in peripheral blood of leukemic mice. Peripheral blood of hTPOtg-RAG2^{-/-}γc^{-/-} mice injected with BV173-ffluc leukemia and T cells was analyzed 10 days after adoptive T cell transfer by FACS for the presence of human T cells. (FIG. 12A) Percent human CD45+CD3+ cells in peripheral blood of mice from mice treated with NT T cells (n=2, black circles), TCR+ T cells (n=5, red squares), TCR+c-MPL+ T cells and PBS injections (n=8, blue triangles up), or TCR+c-MPL+ T cells and rhTPO injections (n=7, blue triangles down). p=0.23, TCR+vs TCR+c-MPL+ and PBS injections; p=0.09, TCR+vs TCR+c-MPL+ and rhTPO injections; t-test. (FIG. 12B) Bar graph for number of engrafted mice per group, with human T cells in mouse blood above the threshold of 1%. Green bar: T cells detected, gray bars: T cells not detected. Percentages above each bar indicate the % of engrafted mice. The numbers within the bar indicate the number of mice per group.

### DETAILED DESCRIPTION OF THE INVENTION

In keeping with long-standing patent law convention, the words “a” and “an” when used in the present specification in concert with the word comprising, including the claims, denote “one or more.” Some embodiments of the disclosure may consist of or consist essentially of one or more elements, method steps, and/or methods of the disclosure. It is contemplated that any method or composition described herein can be implemented with respect to any other method or composition described herein.

As used herein, the term “about” or “approximately” refers to a quantity, level, value, number, frequency, percentage, dimension, size, amount, weight or length that varies by as much as 30, 25, 20, 25, 10, 9, 8, 7, 6, 5, 4, 3, 2 or 1% to a reference quantity, level, value, number, frequency, percentage, dimension, size, amount, weight or length. In particular embodiments, the terms “about” or “approximately” when preceding a numerical value indicates the value plus or minus a range of 15%, 10%, 5%, or 1%.

Throughout this specification, unless the context requires otherwise, the words “comprise”, “comprises” and “comprising” will be understood to imply the inclusion of a stated step or element or group of steps or elements but not the exclusion of any other step or element or group of steps or elements. By “consisting of” is meant including, and limited to, whatever follows the phrase “consisting of.” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that no other elements are optional and may or may not be present depending upon whether or not they affect the activity or action of the listed elements

Reference throughout this specification to “one embodiment,” “an embodiment,” “a particular embodiment,” “a related embodiment,” “a certain embodiment,” “an additional embodiment,” or “a further embodiment” or combinations thereof means that a particular feature, structure or characteristic described in connection with the embodiment is included in at least one embodiment of the present invention. Thus, the appearances of the foregoing phrases in various places throughout this specification are not necessarily all referring to the same embodiment. Furthermore, the particular features, structures, or characteristics may be combined in any suitable manner in one or more embodiments.

Embodiments of the disclosure address current limitations in adoptive cell transfer, particularly that adoptively transferred T cell receptor (TCR)-engineered T cells require co-stimulatory and cytokine signaling to achieve full and sustained activation and that these signals are frequently impaired in cancer patients. To explore this limitation, as described herein, the capacity of TCR T cells manufactured for the treatment of cancer patients was characterized for their ability to achieve full and sustained activation in vitro as well as in vivo in the presence of endogenous or exogenous ligand-dependent activation of co-stimulatory and cytokine signaling pathways. While transgenic TCRs mediate specific target antigen recognition (signal 1), TCR-transgenic T cells lack built-in transgenic co-stimulation (signal 2) to enhance antigen-specific responses. Most engineered T cells rely on host-derived cytokine signals (signal 3) for their sustained in vivo function and persistence, although levels vary in individuals. Also, cytokines may not efficiently reach a tumor site in order to support of adoptively transferred T cell. The hematopoietic growth factor receptor c-MPL (myeloproliferative leukemia) is the receptor for thrombopoietin (TPO) and is expressed in hematopoietic stem cells (HSCs) and progenitor cells of the myelo/megakaryocytic lineage. c-MPL activated pathways overlap with common pathways used by T cell co-stimulatory molecules as well as common γ-chain cytokine receptors (e.g. IL-2, 4, 7, 9, 15, 21). The present disclosure achieves enhancement of T cell persistence and anti-tumor activity in vivo with engineered immune cells that express a transgenic c-MPL receptor that receives both co-stimulatory (signal 2) and cytokine signals (signal 3) upon c-MPL activation through exposure to exogenous TPO or c-MPL agonists, for example.

I. Cells

Immune cells of the disclosure have been modified by the hand of man and are not found in nature. They may be isolated from other cells. Encompassed in the disclosure are cells that recombinantly express c-MPL or functional parts thereof (for example, by expressing exogenously added c-MPL). In specific aspects, the cells are for use in adoptive transfer. The cells may or may not be formulated in a pharmaceutical composition. The cells may be used directly upon manufacture or they may be appropriately stored and/or transported. The cells may be transformed or transfected with one or more vectors as described herein. The recombinant c-MPL-expressing cells may be produced by introducing at least one of the vectors described herein. In certain cases, the presence of the vector in the engineered cell mediates the expression of a c-MPL expression construct, although in some embodiments the c-MPL expression construct is integrated into the genome. That is, nucleic acid molecules or vectors that are introduced into the host cell may either integrate into the genome of the host or it may be maintained extrachromosomally.

In particular embodiments, the cells of the disclosure are immune cells. The immune cells may be of any type, but in specific embodiments they are T cells, including alpha beta and gamma delta T cells and other subpopulations of the Thymus derived lineage such as NKT cells, as well as NK cells, tumor infiltrating lymphocytes, or bone marrow infiltrating lymphocytes, etc.

As used herein, the terms “cell,” “cell line,” and “cell culture” may be used interchangeably. All of these terms also include their progeny, which is any and all subsequent generations. It is understood that all progeny may not be identical due to deliberate or inadvertent mutations. In the context of expressing a heterologous nucleic acid sequence, “host cell” refers to a prokaryotic or eukaryotic cell, and it includes any transformable organism that is capable of replicating a vector and/or expressing a heterologous gene encoded by a vector. A host cell can, and has been, used as a recipient for vectors. A host cell may be “transfected” or “transformed,” which refers to a process by which exogenous nucleic acid is transferred or introduced into the host cell. A transformed cell includes the primary subject cell and its progeny. As used herein, the terms “engineered” and “recombinant” cells or host cells are intended to refer to a cell into which an exogenous nucleic acid sequence, such as, for example, a vector, has been introduced. Therefore, recombinant cells are distinguishable from naturally occurring cells which do not contain a recombinantly introduced nucleic acid.

In certain embodiments, it is contemplated that RNAs or proteinaceous sequences may be co-expressed with other selected RNAs or proteinaceous sequences in the same host cell. Co-expression may be achieved by co-transfecting the host cell with two or more distinct recombinant vectors. Alternatively, a single recombinant vector may be constructed to include multiple distinct coding regions for RNAs, which could then be expressed in host cells transfected with the single vector. In some cases, a vector that encodes exogenous c-MPL is used in the immune cells with another vector that encodes an engineered receptor, for example. In other cases for the immune cells, a vector that encodes exogenous c-MPL is the same vector molecule that encodes an engineered receptor; in such cases, the regulation of expression of exogenous c-MPL may or may not be from the same regulatory element(s) as the regulation of expression of the engineered receptor.

Cells may comprise vectors that employ control sequences that allow it to be replicated and/or expressed in both prokaryotic and eukaryotic cells. One of skill in the art would further understand the conditions under which to incubate all of the above described host cells to maintain them and to permit replication of a vector. Also understood and known are techniques and conditions that would allow large-scale production of vectors, as well as production of the nucleic acids encoded by vectors and their cognate polypeptides, proteins, or peptides.

In embodiments of the disclosure, there is regulation of expression of exogenous c-MPL in cells of the disclosure. The regulation of expression may include constitutive expression of c-MPL, inducible expression of c-MPL, environment-specific expression of c-MPL, or tissue-specific expression of c-MPL, and examples of such promoters are known in the art. Constitutive mammalian promoters include Simian virus 40, Immediate-early Cytomegalovirus, human ubiquitin C, elongation factor 1α-subunit, and Murine Phosphoglycerate Kinase-1, for example.

In particular embodiments, the cells used in the invention are eukaryotic, including mammalian. The cells are particularly human, but can be associated with any animal of interest, particularly domesticated animals, such as equine, bovine, murine, ovine, canine, feline, etc. for use in their respective animal.

The cells can be autologous cells, syngeneic cells, allogeneic cells and even in some cases, xenogeneic cells. The cells may be modified by changing the major histocompatibility complex (“MHC”) profile, by inactivating β2-microglobulin to prevent the formation of functional Class I MHC molecules, inactivation of Class II molecules, providing for expression of one or more MHC molecules, enhancing or inactivating cytotoxic capabilities by enhancing or inhibiting the expression of genes associated with the cytotoxic activity, or the like.

In some instances specific clones or oligoclonal cells may be of interest, where the cells have a particular specificity, such as T cells and B cells having a specific antigen specificity or homing target site specificity, such as survivin, for example.

In particular embodiments the cells that express c-MPL are T cells that have been engineered to express c-MPL or parts thereof. The exemplary T cells may be modified in a way other than recombinantly expressing c-MPL. For example, one may wish to introduce polynucleotides encoding one or more molecules other than c-MPL. In specific cases the polynucleotides encode both chains of a T-cell receptor. For example, in addition to providing for expression of a gene having therapeutic value such as c-MPL and, optionally, another therapeutic gene, in some embodiments the cell is modified to direct the cell to a particular site. The site can include one or more anatomical sites, and in particular embodiments includes non-solid cancers.

In one embodiment, the host cell is a T cell comprising recombinant c-MPL but also comprising at least one engineered TCR or chimeric antigen receptor (CAR). Naturally occurring T cell receptors comprise two subunits, an α-subunit and a β-subunit, each of which is a unique protein produced by recombination event in each T cell's genome. Libraries of TCRs may be screened for their selectivity to particular target antigens. An “engineered TCR” refers to a natural TCR, which has high-avidity and reactivity toward target antigens that is selected, cloned, and/or subsequently introduced into a population of T cells used for adoptive immunotherapy, or it can refer to a receptor that has been produced by the hand of man using recombinant technology.

Cells of the disclosure harboring an exogenous molecule(s) for expression of c-MPL or intended to harbor same may also comprise an engineered T cell receptor including a chimeric antigen receptor (CAR), which generally comprises a tumor-associated antigen (TAA)-binding domain (most commonly a scFv derived from the antigen-binding region of a monoclonal antibody). In addition, the CAR generally comprises an extracellular spacer/hinge region, a transmembrane domain and one or more intracellular signaling domains. The CAR may be first generation, second generation, or third generation, for example. The CAR may be bi-specific, tri-specific, or multi-specific. The TCR and/or CAR, or any engineered receptor of the immune cells, may target one or more antigens associated with hematological malignancies[NRF1]. The TCR and/or CAR, or any engineered receptor of the immune cells, may be specific for EphA2, HER2, GD2, Glypican-3, 5T4, 8H9, αᵥβ₆ integrin, B cell maturation antigen (BCMA) B7-H3, B7-H6, CAIX, CA9, CD19, CD20, CD22, kappa light chain, CD30, CD33, CD38, CD44, CD44v6, CD44v7/8, CD70, CD123, CD138, CD171, CS1, CEA, CSPG4, EGFR, EGFRvIII, EGP2, EGP40, EPCAM, ERBB3, ERBB4, ErbB3/4, FAP, FAR, FBP, fetal AchR, Folate Receptor α, GD3, HLA-AI, HLA-A2, IL11Ra, IL13Ra2, KDR, lambda light chain, Lewis-Y, MCSP, Mesothelin, Muc1, Muc16, NCAM, NKG2D ligands, NY-ESO-1, PRAME, PSCA, PSC1, PSMA, ROR1, Sp17, SURVIVIN, TAG72, TEM1, TEM8, HMW-MAA, VEGF receptors, MAGE-A1, MAGE-A3, MAGE-A4, CT83, SSX2, XIAP, cIAP1, cIAP2, NAIP, and/or Livin, for example. The engineered TCR (or CAR) and c-MPL may be on the same or different vectors. In cases wherein a CAR is employed in the cell, the costimulatory domain(s) may comprise CD3, CD28, 4-1BB, OX40, ICOS, CD27 and so forth. Other examples of engineered receptors include chimeric co-stimulatory receptors, chimeric cytokine receptors, synthetic Notch receptors, drug-inducible receptors, chimeric G-protein coupled receptors, etc.

In some situations, it may be desirable to kill the modified cells, such as when the object is to terminate the treatment, the cells become neoplastic, in research where the absence of the cells after their presence is of interest, and/or another event. For this purpose one can provide for the expression of certain gene products in which one can kill the modified cells under controlled conditions, such as a suicide gene. Suicide genes are known in the art, e.g. the iCaspase9 system in which a modified form of caspase 9 is dimerizable with a small molecule, e.g. AP1903. See, e.g., Straathof et al., Blood 105:4247-4254 (2005).

II. Therapeutic Uses of the Cells

An embodiment of the disclosure relates to the use of engineered immune cells as described herein for the prevention, treatment, or amelioration of a cancerous disease, such as a hematological malignancy. In particular, the pharmaceutical composition of the present disclosure may be particularly useful in treating cancers in which having c-MPL renders the engineered cells of the pharmaceutical composition more effective than if the engineered cells lacked c-MPL. In specific embodiments, cancer cells being treated with pharmaceutical compositions are effectively treated because cells of the pharmaceutical compositions express c-MPL that promotes co-stimulatory and cytokine signaling. In particular embodiments, the cancer is in the form of a hematological malignancy. In particular embodiments, methods comprise use of c-MPL that enhances tumor-targeted T cell function. As shown herein, c-MPL enables tumor-directed TCR+ T cells to become sequential killers by improving immune synapses, co-stimulation and cytokine signals. In addition, c-MPL activation improves in vivo persistence and anti-tumor function of adoptively transferred c-MPL+ TCR-transgenic T cells.

As used herein “treatment” or “treating,” includes any beneficial or desirable effect on the symptoms or pathology of a disease or pathological condition, and may include even minimal reductions in one or more measurable markers of the disease or condition being treated, e.g., cancer. Treatment can involve optionally either the reduction or amelioration of symptoms of the disease or condition, or the delaying of the progression of the disease or condition. “Treatment” does not necessarily indicate complete eradication or cure of the disease or condition, or associated symptoms thereof.

As used herein, “prevent,” and similar words such as “prevented,” “preventing” etc., indicate an approach for preventing, inhibiting, or reducing the likelihood of the occurrence or recurrence of, a disease or condition, e.g., cancer. It also refers to delaying the onset or recurrence of a disease or condition or delaying the occurrence or recurrence of the symptoms of a disease or condition. As used herein, “prevention” and similar words also includes reducing the intensity, effect, symptoms and/or burden of a disease or condition prior to onset or recurrence of the disease or condition.

An individual may be subjected to compositions or methods of the disclosure wherein the individual is at risk for a hematological malignancy. The individual may be at risk because of having one or more known risk factors, such as family or personal history, being a smoker, having one or more genetic markers, exposure to chemicals, and so forth.

Possible indications for administration of the composition(s) of the c-MPL-expressing immune cells are cancerous diseases, including acute lymphoblastic leukemia, acute myelogenous leukemia, chronic lymphocytic leukemia, chronic myelogenous leukemia, acute monocytic leukemia, Hodgkin's lymphoma, non-Hodgkin's lymphoma, breast, prostate, lung, and colon cancers or epithelial cancers/carcinomas such as breast cancer, colon cancer, prostate cancer, head and neck cancer, skin cancer, cancers of the genito-urinary tract, e.g. ovarian cancer, endometrial cancer, cervix cancer and kidney cancer, lung cancer, gastric cancer, cancer of the small intestine, liver cancer, pancreas cancer, gall bladder cancer, cancers of the bile duct, esophagus cancer, cancer of the salivary glands and cancer of the thyroid gland. In particular embodiments, the administration of the composition(s) of the disclosure is useful for all stages and types of cancer, including for minimal residual disease, early cancer, advanced cancer, and/or metastatic cancer and/or refractory cancer, for example.

The disclosure further encompasses co-administration protocols with compounds that are agonists for c-MPL, such as FDA-approved agonists for c-MPL. The clinical regimen for co-administration of the inventive cell(s) may encompass co-administration at the same time or before or after the administration of the other component.

The disclosure further encompasses co-administration protocols with other compounds that are effective against cancer. The clinical regimen for co-administration of the inventive cell(s) may encompass co-administration at the same time, before, or after the administration of the other component. Particular combination therapies include chemotherapy, radiation, surgery, hormone therapy, and/or other types of immunotherapy.

By way of illustration, cancer patients or patients susceptible to cancer or suspected of having cancer may be treated as follows. Cells modified as described herein may be administered to the patient and retained for extended periods of time. The individual may receive one or more administrations of the cells. Illustrative cells include ex vivo expanded T cells that express c-MPL. The cell would be modified at least to express an active part or all of c-MPL and is provided to the individual in need thereof. The cells may be injected directly into the tumor, in some cases, or it may be provided systemically. An exemplary c-MPL nucleotide sequence is in GenBank® Accession No. NM_005373 (SEQ ID NO:1), and an exemplary c-MPL polypeptide sequence is in GenBank® Accession No. NP_005364 (SEQ ID NO:2), both of which are incorporated by reference herein in their entirety. An active part or all of the entire sequence may be incorporated into the cell, although in specific aspects the part of c-MPL that is incorporated includes any domain required for signal transduction, for example. In specific embodiments, the c-MPL transmembrane and/or intracellular domains are utilized in a functionally active fragment of c-MPL. In specific embodiments, the c-MPL fragment comprises sequence that is at least 80, 85, 90, 95, 97, or 99% identical to SEQ ID NO:1 or SEQ ID NO:2, respectively. Any fragment of c-MPL polynucleotide that is employed may comprise at least or no more than 250, 500, 750, 1000, 1250, 1500, 1750, 2000, 2250, 2500, 2750, 3000, 3250, or 3500 nucleotides in length. Any fragment of c-MPL polypeptide that is employed may comprise at least or no more than 50, 75, 100, 125, 150, 175, 200, 225, 250, 275, 300, 325, 350, 375, 400, 425, 450, 475, 500, 525, 550, 575, 600, or 625 amino acids in length.

Another embodiment includes modification of antigen-specific TCR or CAR T cells with c-MPL, where one can activate expression of a protein product to activate the cells. The T cell receptor or CAR could be directed against tumor cells, pathogens, cells mediating autoimmunity, and the like. By providing for activation of the cells, for example, a c-MPL agonist and/or TPO, one could provide for expansion of the modified T cells in response to a ligand. Other uses of the modified T cells would include expression of homing receptors for directing the T cells to specific sites, where cytotoxicity, upregulation of a surface membrane protein of target cells, e.g. endothelial cells, or other biological event would be desired.

III. Introduction of Constructs into Cells

The recombinant c-MPL expression construct(s) can be introduced as one or more DNA molecules or constructs, where there may be at least one marker that will allow for selection of host cells that contain the construct(s). The constructs can be prepared in conventional ways, where the genes and regulatory regions may be isolated, as appropriate, ligated, cloned in an appropriate cloning host, and analyzed by sequencing or other convenient means. Particularly, using PCR, individual fragments including all or portions of a functional unit may be isolated, where one or more mutations may be introduced using “primer repair”, ligation, in vitro mutagensis, etc. as appropriate. The construct(s) once completed and demonstrated to have the appropriate sequences may then be introduced into the host cell by any convenient means. The constructs may be integrated and packaged into non-replicating, defective viral genomes like lentivirus, Adenovirus, Adeno-associated virus (AAV), Herpes simplex virus (HSV), or others, including retroviral vectors, for infection or transduction into cells. The constructs may include viral sequences for transfection, if desired. Alternatively, the construct may be introduced by fusion, electroporation, biolistics, transfection, lipofection, or the like. The host cells may be grown and expanded in culture before introduction of the construct(s), followed by the appropriate treatment for introduction of the construct(s) and integration of the construct(s). The cells are then expanded and screened by virtue of a marker present in the construct. Various markers that may be used successfully include hprt, neomycin resistance, thymidine kinase, hygromycin resistance, etc.

In some instances, c-MPL may be introduced into the cells as an RNA molecule for transient expression. RNA can be delivered to the immune cells of the disclosure by various means including microinjection, electroporation, and lipid-mediated transfection, for example. In particular aspects, introduction of constructs into cells may occur via transposons. An example of a synthetic transposon for use is the Sleeping Beauty transposon that comprises an expression cassette including the c-MPL gene thereof. Alternatively, one may have a target site for homologous recombination, where it is desired that a construct be integrated at a particular locus using materials and methods as are known in the art for homologous recombination. For homologous recombination, one may use either .OMEGA. or O-vectors. See, for example, Thomas and Capecchi, 1987; Mansour, et al., 1988; and Joyner, et al., 1989.

The constructs may be introduced as a single DNA molecule encoding at least c-MPL or parts thereof and optionally another gene, or different DNA molecules having one or more genes. The constructs may be introduced simultaneously or consecutively, each with the same or different markers. In an illustrative example, one construct would contain c-MPL under the control of particular regulatory sequences.

Vectors containing useful elements such as bacterial or yeast origins of replication, selectable and/or amplifiable markers, promoter/enhancer elements for expression in prokaryotes or eukaryotes, etc. that may be used to prepare stocks of construct DNAs and for carrying out transfections are well known in the art, and many are commercially available.

IV. Administration of Cells

The engineered cells that have been modified to express c-MPL or parts thereof are provided to an individual in need thereof. The engineered cells that have been modified to express c-MPL (such as with DNA constructs) may be grown in culture under selective conditions, and cells that are selected as having the construct may then be expanded and further analyzed, using, for example; the polymerase chain reaction for determining the presence of the construct in the host cells. The c-MPL expressing cells may be enriched from the expanded cells using antibody labeling followed by magnetic bead-based separation or other forms of positive selection including column adherence or flow cytometry. Once the modified host cells have been identified, they may then be used as planned, e.g. expanded in culture or introduced into a host individual.

Depending upon the nature of the cells, the cells may be introduced into a host individual, e.g. a mammal, in a wide variety of ways. In specific embodiments the cells hone to the cancer or are modified to hone to the cancer. The number of cells that are employed will depend upon a number of circumstances, the purpose for the introduction, the lifetime of the cells, the protocol to be used, for example, the number of administrations, the ability of the cells to multiply, the stability of the recombinant construct, and the like. The cells may be applied as a dispersion, generally being injected at or near the site of interest. The cells may be in a physiologically-acceptable medium.

In particular embodiments, the route of administration may be retroorbital, intravenous, intraarterial, intraperitoneal, or subcutaneous, for example. Multiple administrations may be by the same route or by different routes.

The DNA introduction need not result in integration in every case. In some situations, transient maintenance of the DNA introduced may be sufficient. In this way, one could have a short-term effect, where cells could be introduced into the host and then turned on after a predetermined time, for example, after the cells have been able to home to a particular site.

The cells may be administered as desired. Depending upon the response desired, the manner of administration, the life of the cells, the number of cells present, various protocols may be employed. The number of administrations will depend upon the factors described herein at least in part.

In particular cases, a plurality of immune cells of the disclosure are delivered to an individual with cancer. In specific embodiments, a single administration is required. In other embodiments, a plurality of administration of cells is required. For example, following a first administration of the engineered immune cells, there may be examination of the individual for the presence or absence of the cancer or for a reduction in the number and/or size of tumors, for example. In the event that the cancer shows a need for further treatment, such as upon tumor growth after the first administration, an additional one or more deliveries of the same engineered immune cells (or, optionally, another type of cancer therapy, including another type of immunotherapy, chemotherapy, surgery and/or radiation) is given to the individual. In some cases, a reduction of tumor size in an individual indicates that the particular immunotherapy is effective, so further administrations of same are provided to the individual.

Determination of appropriate dose levels are routinely performed in the art. In specific cases, a particular dose of immune cells is from 10⁷/m² to 10⁹/m²[NRF2]. In specific embodiments an initial dose of cells is higher than a subsequent dose of cells, whereas in other cases an initial dose of cells is lower than a subsequent dose of cells. The determination of dose may be dependent upon a variety of factors including severity of disease, gender, weight, type of cancer, stage of cancer, overall health of the individual, response to other cancer drug(s), and so forth. In specific embodiments, the following regimen may be employed: dose level 1: 2×10⁷/m²; dose level 2: 1×10⁸/m² based on transduced T cells.

It should be appreciated that the system is subject to variables, such as the cellular response to the ligand, the efficiency of expression and, as appropriate, the activity of the expression product, the particular need of the patient, which may vary with time and circumstances, the rate of loss of the cellular activity as a result of loss of cells or expression activity of individual cells, and the like. Therefore, it is expected that for each individual patient, even if there were universal cells which could be administered to the population at large, each patient would be monitored for the proper dosage for the individual, and such practices of monitoring a patient are routine in the art.

V. Nucleic Acid-Based Expression Systems

In aspects of the disclosure, there are cells that express exogenously provided c-MPL or parts thereof, wherein the c-MPL expression is produced from recombinant DNA in the cells. The c-MPL coding sequence may be provided on a vector, including an expression vector, for example. Other gene products (such as an engineered receptor, including a TCR, CAR and/or an engager molecule) may be expressed from the same expression vector, or they may be present in a cell on separate vector(s) from the c-MPL.

A. Vectors

The term “vector” is used to refer to a carrier nucleic acid molecule into which a nucleic acid sequence can be inserted for introduction into a cell where it can be replicated. A nucleic acid sequence can be “exogenous,” which means that it is foreign to the cell into which the vector is being introduced or that the sequence is homologous to a sequence in the cell but in a position within the host cell nucleic acid in which the sequence is ordinarily not found. Vectors include plasmids, cosmids, viruses (bacteriophage, animal viruses, and plant viruses), and artificial chromosomes (e.g., BACs, YACs). One of skill in the art would be well equipped to construct a vector through standard recombinant techniques (see, for example, Maniatis et al., 1988 and Ausubel et al., 1994, both incorporated herein by reference).

Vectors can include a multiple cloning site (MCS), which is a nucleic acid region that contains multiple restriction enzyme sites, any of which can be used in conjunction with standard recombinant technology to digest the vector. “Restriction enzyme digestion” refers to catalytic cleavage of a nucleic acid molecule with an enzyme that functions only at specific locations in a nucleic acid molecule. Many of these restriction enzymes are commercially available. Use of such enzymes is widely understood by those of skill in the art. Frequently, a vector is linearized or fragmented using a restriction enzyme that cuts within the MCS to enable exogenous sequences to be ligated to the vector. “Ligation” refers to the process of forming phosphodiester bonds between two nucleic acid fragments, which may or may not be contiguous with each other. Techniques involving restriction enzymes and ligation reactions are well known to those of skill in the art of recombinant technology.

The term “expression vector” refers to any type of genetic construct comprising a nucleic acid coding for a RNA capable of being transcribed. In some cases, RNA molecules are then translated into a protein, polypeptide, or peptide. In other cases, these sequences are not translated, for example, in the production of antisense molecules or ribozymes. Expression vectors can contain a variety of “control sequences,” which refer to nucleic acid sequences necessary for the transcription and possibly translation of an operably linked coding sequence in a particular host cell. Splicing sites, termination signals, origins of replication, and selectable markers may also be employed. In addition to control sequences that govern transcription and translation, vectors and expression vectors may contain nucleic acid sequences that serve other functions as well and are described infra.

B. Promoters and Enhancers

A “promoter” is a control sequence that is a region of a nucleic acid sequence at which initiation and rate of transcription are controlled. It may contain genetic elements at which regulatory proteins and molecules may bind, such as RNA polymerase and other transcription factors, to initiate the specific transcription of a nucleic acid sequence. The phrases “operatively positioned,” “operatively linked,” “under control,” and “under transcriptional control” mean that a promoter is in a correct functional location and/or orientation in relation to a nucleic acid sequence to control transcriptional initiation and/or expression of that sequence.

A promoter generally comprises a sequence that functions to position the start site for RNA synthesis. The best known example of this is the TATA box, but in some promoters lacking a TATA box, such as, for example, the promoter for the mammalian terminal deoxynucleotidyl transferase gene and the promoter for the SV40 late genes, a discrete element overlying the start site itself helps to fix the place of initiation. Additional promoter elements regulate the frequency of transcriptional initiation. Typically, these are located in the region 30-110 bp upstream of the start site, although a number of promoters have been shown to contain functional elements downstream of the start site as well. To bring a coding sequence “under the control of” a promoter, one positions the 5′ end of the transcription initiation site of the transcriptional reading frame “downstream” of (i.e., 3′ of) the chosen promoter. The “upstream” promoter stimulates transcription of the DNA and promotes expression of the encoded RNA.

The spacing between promoter elements frequently is flexible, so that promoter function is preserved when elements are inverted or moved relative to one another. In the tk promoter, the spacing between promoter elements can be increased to 50 bp apart before activity begins to decline. Depending on the promoter, it appears that individual elements can function either cooperatively or independently to activate transcription. A promoter may or may not be used in conjunction with an “enhancer,” which refers to a cis-acting regulatory sequence involved in the transcriptional activation of a nucleic acid sequence.

A promoter may be one naturally associated with a nucleic acid sequence, as may be obtained by isolating the 5′ non-coding sequences located upstream of the coding segment and/or exon. Such a promoter can be referred to as “endogenous.” Similarly, an enhancer may be one naturally associated with a nucleic acid sequence, located either downstream or upstream of that sequence. Alternatively, certain advantages will be gained by positioning the coding nucleic acid segment under the control of a recombinant or heterologous promoter, which refers to a promoter that is not normally associated with a nucleic acid sequence in its natural environment. A recombinant or heterologous enhancer refers also to an enhancer not normally associated with a nucleic acid sequence in its natural environment. Such promoters or enhancers may include promoters or enhancers of other genes, and promoters or enhancers isolated from any other virus, or prokaryotic or eukaryotic cell, and promoters or enhancers not “naturally occurring,” i.e., containing different elements of different transcriptional regulatory regions, and/or mutations that alter expression. For example, promoters that are most commonly used in recombinant DNA construction include the β-lactamase (penicillinase), lactose and tryptophan (trp) promoter systems. In addition to producing nucleic acid sequences of promoters and enhancers synthetically, sequences may be produced using recombinant cloning and/or nucleic acid amplification technology, including PCR™, in connection with the compositions disclosed herein (see U.S. Pat. Nos. 4,683,202 and 5,928,906, each incorporated herein by reference). Furthermore, it is contemplated the control sequences that direct transcription and/or expression of sequences within non-nuclear organelles such as mitochondria, chloroplasts, and the like, can be employed as well.

It will be important to employ a promoter and/or enhancer that effectively directs the expression of the DNA segment in the organelle, cell type, tissue, organ, or organism chosen for expression. Those of skill in the art of molecular biology generally know the use of promoters, enhancers, and cell type combinations for protein expression, (see, for example Sambrook et al. 1989, incorporated herein by reference). The promoters employed may be constitutive, tissue-specific, inducible, and/or useful under the appropriate conditions to direct high level expression of the introduced DNA segment, such as is advantageous in the large-scale production of recombinant proteins and/or peptides. The promoter may be heterologous or endogenous. In specific embodiments, the c-MPL expression is under control of an inducible or tissue-specific promoter. Tissue-specific promoters are known in the art, but in specific embodiments the tissue-specificity is tailored to the tissue in which the cancer is located. The identity of tissue-specific promoters or elements, as well as assays to characterize their activity, is well known to those of skill in the art, such as hypoxia-inducible promoters.

Additionally any promoter/enhancer combination could also be used to drive expression. Use of a T3, T7 or SP6 cytoplasmic expression system is another possible embodiment. Eukaryotic cells can support cytoplasmic transcription from certain bacterial promoters if the appropriate bacterial polymerase is provided, either as part of the delivery complex or as an additional genetic expression construct.

A specific initiation signal also may be required for efficient translation of coding sequences. These signals include the ATG initiation codon or adjacent sequences. Exogenous translational control signals, including the ATG initiation codon, may need to be provided. One of ordinary skill in the art would readily be capable of determining this and providing the necessary signals.

In certain embodiments of the disclosure, the use of internal ribosome entry sites (IRES) or 2A elements are used to create multigene, or polycistronic, messages, and these may be used in the disclosure.

C. Plasmid Vectors

In certain embodiments, a plasmid vector is contemplated for use to transform a host cell. In general, plasmid vectors containing replicon and control sequences which are derived from species compatible with the host cell are used in connection with these hosts. The vector ordinarily carries a replication site, as well as marking sequences which are capable of providing phenotypic selection in transformed cells. In a non-limiting example, E. coli is often transformed using derivatives of pBR322, a plasmid derived from an E. coli species. pBR322 contains genes for ampicillin and tetracycline resistance and thus provides easy means for identifying transformed cells. The pBR plasmid, or other microbial plasmid or phage must also contain, or be modified to contain, for example, promoters which can be used by the microbial organism for expression of its own proteins.

In addition, phage vectors containing replicon and control sequences that are compatible with the host microorganism can be used as transforming vectors in connection with these hosts. For example, the phage lambda GEM™-11 may be utilized in making a recombinant phage vector which can be used to transform host cells, such as, for example, E. coli LE392.

Further useful plasmid vectors include pIN vectors (Inouye et al., 1985); and pGEX vectors, for use in generating glutathione S-transferase (GST) soluble fusion proteins for later purification and separation or cleavage. Other suitable fusion proteins are those with 3-galactosidase, ubiquitin, and the like.

Bacterial host cells, for example, E. coli, comprising the expression vector, are grown in any of a number of suitable media, for example, LB. The expression of the recombinant protein in certain vectors may be induced, as would be understood by those of skill in the art, by contacting a host cell with an agent specific for certain promoters, e.g., by adding IPTG to the media or by switching incubation to a higher temperature. After culturing the bacteria for a further period, generally of between 2 and 24 h, the cells are collected by centrifugation and washed to remove residual media.

D. Viral Vectors

The ability of certain viruses to infect cells or enter cells via receptor-mediated endocytosis, and to integrate into host cell genome and express viral genes stably and efficiently have made them attractive candidates for the transfer of foreign nucleic acids into cells (e.g., mammalian cells). Components of the present disclosure may be a viral vector that encodes c-MPL or parts thereof. Non-limiting examples of virus vectors that may be used to deliver a nucleic acid of the present disclosure are described below.

1. Retroviral Vectors

Retroviruses are useful as delivery vectors because of their ability to integrate their genes into the host genome, transferring a large amount of foreign genetic material, infecting a broad spectrum of species and cell types and of being packaged in special cell-lines (Miller, 1992).

In order to construct a c-MPL retroviral vector, a nucleic acid (e.g., one encoding part or all of c-MPL) is inserted into the viral genome in the place of certain viral sequences to produce a virus that is replication-defective. In order to produce virions, a packaging cell line containing the gag, pol, and env genes but without the LTR and packaging components is constructed (Mann et al., 1983). When a recombinant plasmid containing a cDNA, together with the retroviral LTR and packaging sequences is introduced into a special cell line (e.g., by calcium phosphate precipitation for example), the packaging sequence allows the RNA transcript of the recombinant plasmid to be packaged into viral particles, which are then secreted into the culture media (Nicolas and Rubenstein, 1988; Temin, 1986; Mann et al., 1983). The media containing the recombinant retroviruses is then collected, optionally concentrated, and used for gene transfer. Retroviral vectors are able to infect a broad variety of cell types. However, integration and stable expression require the division of host cells (Paskind et al., 1975).

Lentiviruses are complex retroviruses, which, in addition to the common retroviral genes gag, pol, and env, contain other genes with regulatory or structural function. Lentiviral vectors are well known in the art (see, for example, Naldini et al., 1996; Zufferey et al., 1997; Blomer et al., 1997; U.S. Pat. Nos. 6,013,516 and 5,994,136). Some examples of lentivirus include the Human Immunodeficiency Viruses: HIV-1, HIV-2 and the Simian Immunodeficiency Virus: SIV. Lentiviral vectors have been generated by multiply attenuating the HIV virulence genes, for example, the genes env, vif, vpr, vpu and nef are deleted making the vector biologically safe.

Recombinant lentiviral vectors are capable of infecting non-dividing cells and can be used for both in vivo and ex vivo gene transfer and expression of nucleic acid sequences. For example, recombinant lentivirus capable of infecting a non-dividing cell wherein a suitable host cell is transfected with two or more vectors carrying the packaging functions, namely gag, pol and env, as well as rev and tat is described in U.S. Pat. No. 5,994,136, incorporated herein by reference. One may target the recombinant virus by linkage of the envelope protein with an antibody or a particular ligand for targeting to a receptor of a particular cell-type. By inserting a sequence (including a regulatory region) of interest into the viral vector, along with another gene which encodes the ligand for a receptor on a specific target cell, for example, the vector is now target-specific.

2. Adenoviral Vectors

A particular method for delivery of the nucleic acid involves the use of an adenovirus expression vector. Although adenovirus vectors are known to have a low capacity for integration into genomic DNA, this feature is counterbalanced by the high efficiency of gene transfer afforded by these vectors. “Adenovirus expression vector” is meant to include those constructs containing adenovirus sequences sufficient to (a) support packaging of the construct and (b) to ultimately express a tissue or cell-specific construct that has been cloned therein. Knowledge of the genetic organization or adenovirus, a 36 kb, linear, double-stranded DNA virus, allows substitution of large pieces of adenoviral DNA with foreign sequences up to 7 kb (Grunhaus and Horwitz, 1992).

3. AAV Vectors

The nucleic acid may be introduced into the cell using adenovirus assisted transfection. Increased transfection efficiencies have been reported in cell systems using adenovirus coupled systems (Kelleher and Vos, 1994; Cotten et al., 1992; Curiel, 1994). AAV has a broad host range for infectivity (Tratschin et al., 1984; Laughlin et al., 1986; Lebkowski et al., 1988; McLaughlin et al., 1988). Details concerning the generation and use of rAAV vectors are described in U.S. Pat. Nos. 5,139,941 and 4,797,368, each incorporated herein by reference.

4. Other Viral Vectors

Other viral vectors may be employed as vaccine constructs in the present disclosure. Vectors derived from viruses such as vaccinia virus (Ridgeway, 1988; Baichwal and Sugden, 1986; Coupar et al., 1988), sindbis virus, cytomegalovirus and herpes simplex virus may be employed. They offer several attractive features for various mammalian cells (Friedmann, 1989; Ridgeway, 1988; Baichwal and Sugden, 1986; Coupar et al., 1988; Horwich et al., 1990).

E. Delivery Using Modified Viruses

A nucleic acid to be delivered may be housed within an infective virus that has been engineered to express a specific binding ligand. The virus particle will thus bind specifically to the cognate receptors of the target cell and deliver the contents to the cell.

F. Vector Delivery and Cell Transformation

Suitable methods for nucleic acid delivery for transfection or transduction of cells are known to one of ordinary skill in the art. Such methods include, but are not limited to, direct delivery of DNA such as by ex vivo transfection, by injection, and so forth. Through the application of techniques known in the art, cells may be stably or transiently transformed.

G. Ex Vivo Transformation

Methods for transfecting eukaryotic cells and tissues removed from an organism in an ex vivo setting are known to those of skill in the art. Thus, it is contemplated that cells or tissues may be removed and transfected ex vivo using c-MPL or other nucleic acids of the present disclosure. In particular aspects, the transplanted cells or tissues may be placed into an organism. In preferred facets, a nucleic acid is expressed in the transplanted cells.

VI. Kits

Any of the compositions described herein may be comprised in a kit. In a non-limiting example, one or more immune cells for use in cell therapy that harbors recombinantly expressed c-MPL and/or the reagents to generate and/or activate one or more cells for use in cell therapy that harbors recombinantly expressed c-MPL may be comprised in a kit. The kit components are provided in suitable container means. In specific embodiments, the kits comprise recombinant engineering reagents, such as vectors, primers, enzymes (restriction enzymes, ligase, polymerases, etc.), buffers, nucleotides, etc.

Some components of the kits may be packaged either in aqueous media or in lyophilized form. The container means of the kits will generally include at least one vial, test tube, flask, bottle, syringe or other container means, into which a component may be placed, and preferably, suitably aliquoted. Where there are more than one component in the kit, the kit also will generally contain a second, third or other additional container into which the additional components may be separately placed. However, various combinations of components may be comprised in a vial. The kits of the present disclosure also will typically include a means for containing the components in close confinement for commercial sale. Such containers may include injection or blow-molded plastic containers into which the desired vials are retained.

When the components of the kit are provided in one and/or more liquid solutions, the liquid solution is an aqueous solution, with a sterile aqueous solution being particularly useful. In some cases, the container means may itself be a syringe, pipette, and/or other such like apparatus, from which the formulation may be applied to an infected area of the body, injected into an animal, and/or even applied to and/or mixed with the other components of the kit.

However, the components of the kit may be provided as dried powder(s). When reagents and/or components are provided as a dry powder, the powder can be reconstituted by the addition of a suitable solvent. It is envisioned that the solvent may also be provided in another container means. The kits may also comprise a second container means for containing a sterile, pharmaceutically acceptable buffer and/or other diluent.

In particular embodiments of the disclosure, cells that are to be used for cell therapy are provided in a kit, and in some cases the cells are essentially the sole component of the kit. The kit may comprise instead or in addition reagents and materials to make the cell recombinant for c-MPL. In specific embodiments, the reagents and materials include primers for amplifying c-MPL, nucleotides, suitable buffers or buffer reagents, salt, and so forth, and in some cases the reagents include vectors and/or DNA that encodes c-MPL and/or regulatory elements therefor.

In particular embodiments, there are one or more apparatuses in the kit suitable for extracting one or more samples from an individual. The apparatus may be a syringe, scalpel, and so forth.

In some cases of the disclosure, the kit, in addition to cell therapy embodiments, also includes a second cancer therapy, such as chemotherapy, hormone therapy, and/or immunotherapy, for example. The kit(s) may be tailored to a particular cancer for an individual and comprise respective second cancer therapies for the individual.

In some cases of the disclosure, the cell in the kit may be modified to express a therapeutic molecule other than c-MPL. The other therapeutic molecule may be of any kind, but in specific embodiments, the therapeutic molecule is an engineered TCR, for example. The kit may include one or more reagents to generate the engineered TCR, including vectors, primers, enzymes, etc.

In some cases, the kit, in addition to cell therapy embodiments, may also include a small molecule ligand to activate c-MPL-expressing cells, such as an agonist for c-MPL, for example. The kit(s) may be tailored to a particular cancer for an individual and comprise respective adjuvant therapies for the individual.

VII. Combination Therapy

In certain embodiments of the disclosure, methods of the present disclosure for clinical aspects are combined with other agents effective in the treatment of hyperproliferative disease, such as anti-cancer agents (which may also be referred to as a cancer therapy). An “anti-cancer” agent is capable of negatively affecting cancer in a subject, for example, by killing cancer cells, inducing apoptosis in cancer cells, reducing the growth rate of cancer cells, reducing the incidence or number of metastases, reducing tumor size, inhibiting tumor growth, reducing the blood supply to a tumor or cancer cells, promoting an immune response against cancer cells or a tumor, preventing or inhibiting the progression of cancer, or increasing the lifespan of a subject with cancer. More generally, these other compositions would be provided in a combined amount effective to kill or inhibit proliferation of the cell. This process may involve contacting the cancer cells with the expression construct and the agent(s) or multiple factor(s) at the same time. This may be achieved by contacting the cell with a single composition or pharmacological formulation that includes both agents, or by contacting the cell with two distinct compositions or formulations, at the same time, wherein one composition includes the expression construct and the other includes the second agent(s).

Tumor cell resistance to chemotherapy and radiotherapy agents represents a major problem in clinical oncology. One goal of current cancer research is to find ways to improve the efficacy of chemo- and radiotherapy by combining it with gene therapy. For example, the herpes simplex-thymidine kinase (HStk) gene, when delivered to brain tumors by a retroviral vector system, successfully induced susceptibility to the antiviral agent ganciclovir (Culver, et al., 1992). In the context of the present disclosure, it is contemplated that cell therapy could be used similarly in conjunction with chemotherapeutic, radiotherapeutic, or immunotherapeutic intervention, in addition to other pro-apoptotic or cell cycle regulating agents.

Alternatively, the present inventive therapy may precede or follow the other agent treatment by intervals ranging from minutes to weeks. In embodiments where the other agent and present disclosure are applied separately to the individual, one would generally ensure that a significant period of time did not expire between the time of each delivery, such that the agent and inventive therapy would still be able to exert an advantageously combined effect on the cell. In such instances, it is contemplated that one may contact the cell with both modalities within about 12-24 h of each other and, more preferably, within about 6-12 h of each other. In some situations, it may be desirable to extend the time period for treatment significantly, however, where several days (2, 3, 4, 5, 6 or 7) to several weeks (1, 2, 3, 4, 5, 6, 7 or 8) lapse between the respective administrations.

Various combinations may be employed, present engineered immune cells is “A” and the secondary agent, such as radio- or chemotherapy, is “B”:

A/B/A B/A/B B/B/A A/A/B A/B/B B/A/A A/B/B/B B/A/B/B
B/B/B/A B/B/A/B A/A/B/B A/B/A/B A/B/B/A B/B/A/A
B/A/B/A B/A/A/B A/A/A/B B/A/A/A A/B/A/A A/A/B/A

It is expected that the treatment cycles would be repeated as necessary. It also is contemplated that various standard therapies, as well as surgical intervention, may be applied in combination with the inventive cell therapy.

A. Chemotherapy

Cancer therapies also include a variety of combination therapies with both chemical and radiation based treatments. Combination anti-cancer agents include, for example, acivicin; aclarubicin; acodazole hydrochloride; acronine; adozelesin; aldesleukin; altretamine; ambomycin; ametantrone acetate; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; celecoxib (COX-2 inhibitor); chlorambucil; cirolemycin; cisplatin; cladribine; crisnatol mesylate; cyclophosphamide; cytarabine; dacarbazine; dactinomycin; daunorubicin hydrochloride; decitabine; dexormaplatin; dezaguanine; dezaguanine mesylate; diaziquone; docetaxel; doxorubicin; doxorubicin hydrochloride; droloxifene; droloxifene citrate; dromostanolone propionate; duazomycin; edatrexate; eflomithine hydrochloride; elsamitrucin; enloplatin; enpromate; epipropidine; epirubicin hydrochloride; erbulozole; esorubicin hydrochloride; estrarnustine; estramustine phosphate sodium; etanidazole; etoposide; etoposide phosphate; etoprine; fadrozole hydrochloride; fazarabine; fenretinide; floxuridine; fludarabine phosphate; fluorouracil; fluorocitabine; fosquidone; fostriecin sodium; gemcitabine; gemcitabine hydrochloride; hydroxyurea; idarubicin hydrochloride; ifosfamide; ilmofosine; iproplatin; irinotecan; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazole; nogalamycin; ormaplatin; oxisuran; paclitaxel; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; taxotere; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; zorubicin hydrochloride; 20-epi-1,25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorins; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidenmin B; deslorelin; dexamethasone; dexifosfamide; dexrazoxane; dexverapamil; diaziquone: didemnin B; didox; diethylnorspermine; dihydro-5-azacytidine; dihydrotaxol, 9-; dioxamycin; diphenyl spiromustine; docetaxel; docosanol; dolasetron; doxifluridine; doxorubicin; droloxifene; dronabinol; duocarmycin SA; ebselen; ecomustine; edelfosine; edrecolomab; eflornithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imatinib (e.g., GLEEVEC®), imiquimod; immunostimulant peptides; insulin-like growth factor-1 receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mitoguazone; mitolactol; mitomycin analogues; mitonafide; mitotoxin fibroblast growth factor-saporin; mitoxantrone; mofarotene; molgramostim; Erbitux, human chorionic gonadotrophin; monophosphoryl lipid A+myobacterium cell wall sk; mopidamol; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin: neridronic acid; nilutamide; nisamycin; nitric oxide modulators; nitroxide antioxidant; nitrullyn; oblimersen (GENASENSE®); O.sup.6-benzylguanine; octreotide; okicenone; oligonucleotides; onapristone; ondansetron; ondansetron; oracin; oral cytokine inducer; ormaplatin; osaterone; oxaliplatin; oxaunomycin; paclitaxel; paclitaxel analogues; paclitaxel derivatives; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylene conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; sizofuran; sobuzoxane; sodium borocaptate; sodium phenylacetate; solverol; somatomedin binding protein; sonermin; sparfosic acid; spicamycin D; spiromustine; splenopentin; spongistatin 1; squalamine; stipiamide; stromelysin inhibitors; sulfinosine; superactive vasoactive intestinal peptide antagonist; suradista; suramin; swainsonine; tallimustine; tamoxifen methiodide; tauromustine; tazarotene; tecogalan sodium; tegafur; tellurapyrylium; telomerase inhibitors; temoporfin; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; velaresol; veramine; verdins; verteporfin; vinorelbine; vinxaltine; vitaxin; vorozole; zanoterone; zeniplatin; zilascorb; and zinostatin stimalamer, or any analog or derivative variant of the foregoing. In specific embodiments, chemotherapy is employed in conjunction with the disclosure, for example before, during and/or after administration of the disclosure. Exemplary chemotherapeutic agents include at least dacarbazine (also termed DTIC), temozolimide, paclitaxel, cisplatin, carmustine, fotemustine, vindesine, vincristine, or bleomycin.

B. Radiotherapy

Other factors that cause DNA damage and have been used extensively include what are commonly known as Trays, X-rays, and/or the directed delivery of radioisotopes to tumor cells. Other forms of DNA damaging factors are also contemplated such as microwaves and UV-irradiation. It is most likely that all of these factors affect a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 weeks), to single doses of 2000 to 6000 roentgens. Dosage ranges for radioisotopes vary widely, and depend on the half-life of the isotope, the strength and type of radiation emitted, and the uptake by the neoplastic cells.

The terms “contacted” and “exposed,” when applied to a cell, are used herein to describe the process by which a therapeutic construct and a chemotherapeutic or radiotherapeutic agent are delivered to a target cell or are placed in direct juxtaposition with the target cell. To achieve cell killing or stasis, both agents are delivered to a cell in a combined amount effective to kill the cell or prevent it from dividing.

C. Immunotherapy

Immunotherapies other than the c-MPL-bearing cells may be employed in addition to the c-MPL-bearing cells. Immunotherapies generally rely on the use of immune effector cells and molecules to target and destroy cancer cells. The immune effector may be, for example, an antibody specific for some marker on the surface of a tumor cell. The antibody alone may serve as an effector of therapy or it may recruit other cells to actually affect cell killing. The antibody also may be conjugated to a drug or toxin (chemotherapeutic, radionuclide, ricin A chain, cholera toxin, pertussis toxin, etc.) and serve merely as a targeting agent. Alternatively, the effector may be a lymphocyte carrying a surface molecule that interacts, either directly or indirectly, with a tumor cell target. Various effector cells include cytotoxic T cells and NK cells.

Immunotherapy could thus be used as part of a combined therapy, in conjunction with the present disclosure. The general approach for combined therapy is discussed below. Generally, the tumor cell must bear some marker that is amenable to targeting, i.e., is not present on the majority of other cells. Many tumor markers exist and any of these may be suitable for targeting in the context of the present disclosure. Common tumor markers include EphA2, HER2, GD2, Glypican-3, 5T4, 8H9, αᵥβ₆ integrin, B cell maturation antigen (BCMA) B7-H3, B7-H6, CAIX, CA9, CD19, CD20, CD22, kappa light chain, CD30, CD33, CD38, CD44, CD44v6, CD44v7/8, CD70, CD123, CD138, CD171, CS1, CEA, CSPG4, EGFR, EGFRvIII, EGP2, EGP40, EPCAM, ERBB3, ERBB4, ErbB3/4, FAP, FAR, FBP, fetal AchR, Folate Receptor α, GD3, HLA-AI, HLA-A2, IL11Ra, IL13Ra2, KDR, Lambda, Lewis-Y, MCSP, Mesothelin, Muc1, Muc16, NCAM, NKG2D ligands, NY-ESO-1, PRAME, PSCA, PSC1, PSMA, ROR1, Sp17, SURVIVIN, TAG72, TEM1, TEM8, carcinoembryonic antigen, HMW-MAA, VEGF receptors, MAGE-A1, MAGE-A3, MAGE-A4, CT83, SSX2, XIAP, cIAP1, cIAP2, NAIP, and/or Livin, for example.

Immunotherapy may include interleukin-2 (IL-2) or interferon (IFN), for example. In certain embodiments, the immunotherapy is an antibody against a Notch pathway ligand or receptor, e.g., an antibody against DLL4, Notch1, Notch2/3, Fzd7, or Wnt. In certain other embodiments, the immunotherapy is an antibody against r-spondin (RSPO) 1, RSPO2, RSPO3 or RSPO4.

D. Genes

In yet another embodiment, the secondary treatment is a gene therapy in which a therapeutic polynucleotide is administered before, after, or at the same time as the clinical embodiments of the present disclosure. A variety of expression products are encompassed within the disclosure, including inducers of cellular proliferation, inhibitors of cellular proliferation, or regulators of programmed cell death.

E. Surgery

Approximately 60% of persons with cancer will undergo surgery of some type, which includes preventative, diagnostic or staging, curative and palliative surgery. Curative surgery is a cancer treatment that may be used in conjunction with other therapies, such as the treatment of the present disclosure, chemotherapy, radiotherapy, hormonal therapy, gene therapy, immunotherapy and/or alternative therapies.

Curative surgery includes resection in which all or part of cancerous tissue is physically removed, excised, and/or destroyed. Tumor resection refers to physical removal of at least part of a tumor. In addition to tumor resection, treatment by surgery includes laser surgery, cryosurgery, electrosurgery, and microscopically controlled surgery (Mohs' surgery). It is further contemplated that the present disclosure may be used in conjunction with removal of superficial cancers, precancers, or incidental amounts of normal tissue.

Upon excision of part of all of cancerous cells, tissue, or tumor, a cavity may be formed in the body. Treatment may be accomplished by perfusion, direct injection or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

### EXAMPLES

The following examples are included to demonstrate preferred embodiments of the invention. It should be appreciated by those of skill in the art that the techniques disclosed in the examples that follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### C-MPL Expression in Polyclonal T Cells Leads to Agonist-Dependent T Cell Expansion and Persistence

Adoptively transferred T cell receptor (TCR)-engineered T-cells depend on host-derived co-stimulation and cytokine signals for their full and sustained activation. However, in cancer patients both signals are frequently impaired. The present disclosure provides a novel strategy that combines both essential signals in one transgene by expressing the non-lymphoid hematopoietic growth factor receptor c-MPL (myeloproliferative leukemia), the receptor for thrombopoietin (TPO), in T-cells. C-MPL signaling activates pathways shared with conventional co-stimulatory and cytokine receptor signaling. Thus, it was considered that host-derived TPO, present in the tumor microenvironment, or FDA-approved pharmacological c-MPL agonists could deliver both signals to c-MPL engineered TCR-transgenic T-cells. As shown herein, c-MPL+ polyclonal T cells expand and proliferate in response to TPO, and persist longer after adoptive transfer in immunodeficient human TPO-transgenic mice. In TCR-transgenic T-cells, c-MPL activation enhances anti-tumor function, T-cell expansion, and cytokine production and preserves a central memory phenotype. C-MPL signaling also enables sequential tumor cell killing, enhances the formation of effective immune synapses, and improves anti-leukemic activity in vivo in a leukemia xenograft model. The type I interferon pathway was identified as a molecular mechanism by which c-MPL mediates immune-stimulation in T cells. Thus, the present disclosure provides a novel immunotherapeutic strategy using c-MPL enhanced transgenic T cells responding to either endogenously-produced TPO (a microenvironment factor in hematologic malignancies, for example) or c-MPL-targeted pharmacological agent(s).

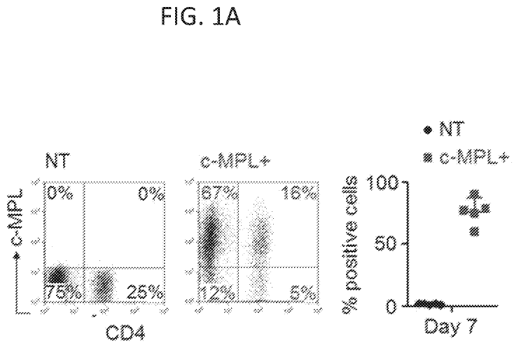

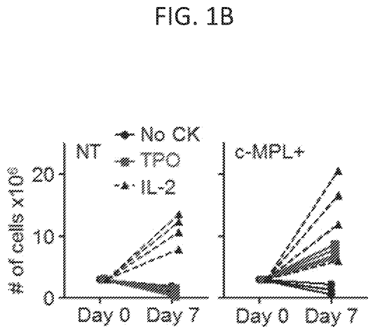

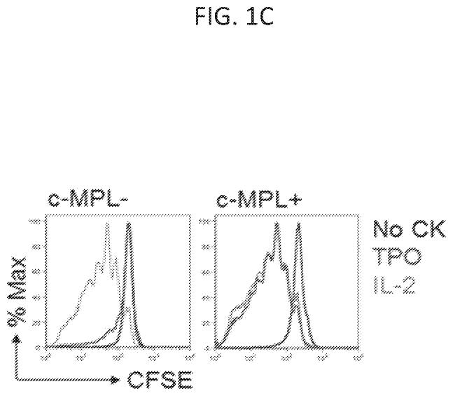

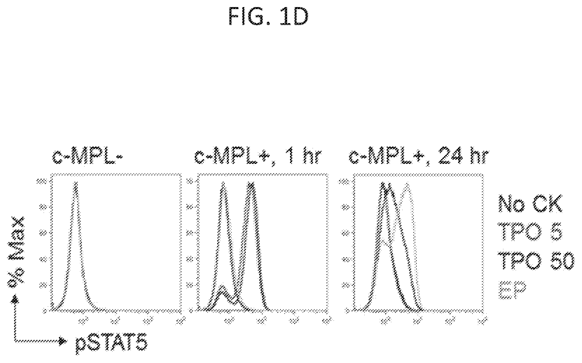

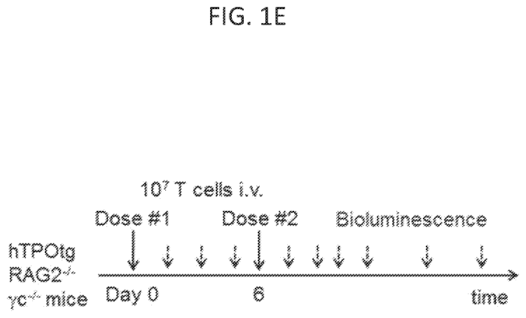

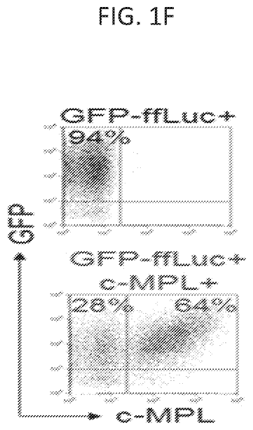

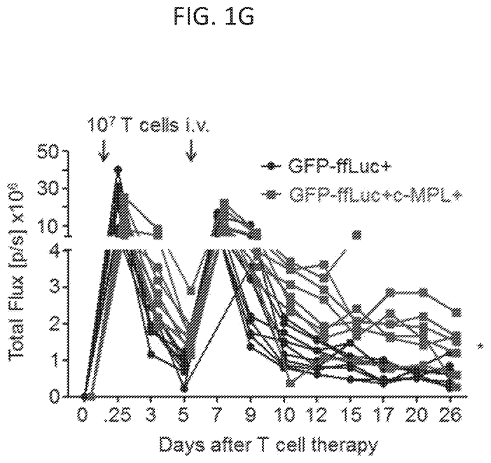

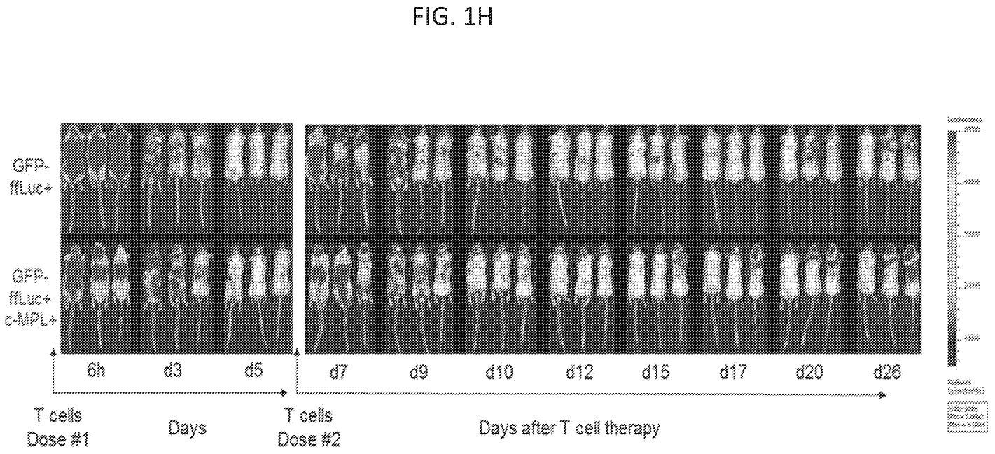

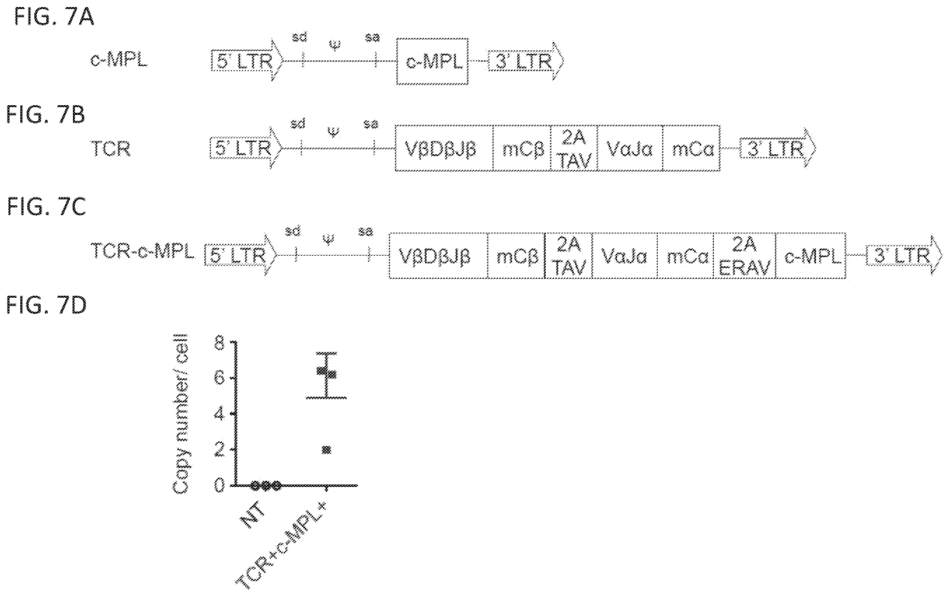

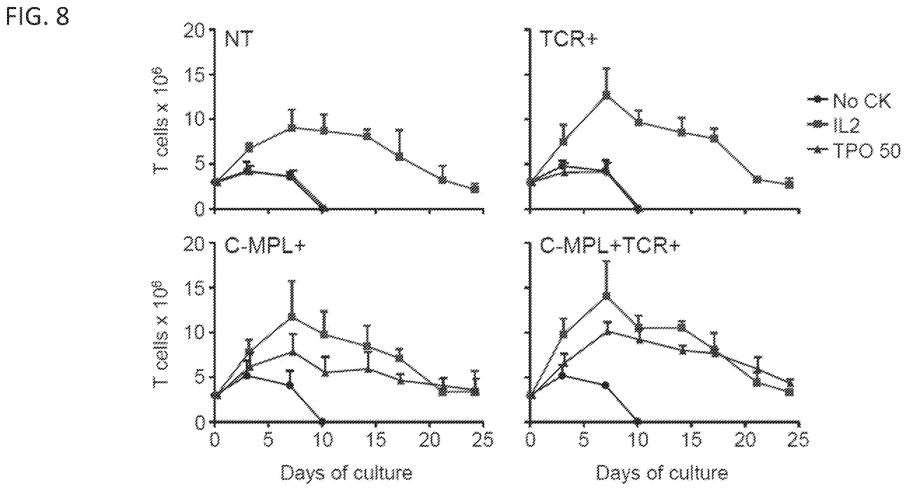

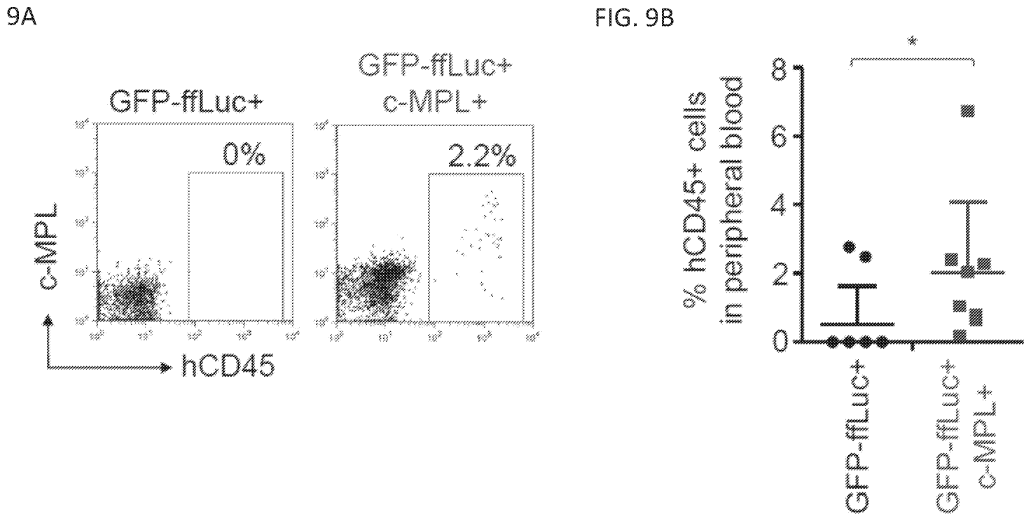

To test whether c-MPL can be expressed efficiently in polyclonal T cells, a retroviral vector was constructed encoding the c-MPL gene (FIG. 7A) and activated polyclonal T cells were transduced. Transduction efficiencies were high in both CD4+ and CD8+ T cells (1.6±0.5% non-transduced (NT) vs 76.3±10.9% c-MPL transduced (c-MPL+) cells, mean±SD, n=5, p<0.001, FIG. 1A). C-MPL+ T cells expanded (FIG. 1B) and proliferated (FIG. 1C) in the presence of recombinant human thrombopoietin (TPO) during the first week at a similar rate to cells cultured in IL2 (p=NS), while c-MPL− control cells did not (0.89±0.61×10⁶NT vs 7.44±0.99×10⁶ c-MPL+ cells in TPO by day 7 from 3×10⁶ cells, mean±SD, n=4, p<0.001, FIG. 1B). Peak expansion was observed after 1 week of culture, followed by a steady decline in T cell numbers in the absence of repeated TCR stimulation (FIG. 8). No growth factor independent cell growth was observed. Activation of c-MPL downstream signaling was assessed by phosflow for pSTAT5 protein (FIG. 1D). STATS phosphorylation occurred in c-MPL+ T cells in response to rhTPO at 5 and 50 ng/ml, or with the small molecule TPO mimetic drug eltrombopag (EP, 0.1 μg/ml). The in vivo persistence of polyclonal c-MPL+ T cells was next investigated under homeostatic steady-state cytokine conditions in immune-deficient human-TPO transgenic (hTPOtg-RAG2^{-/-}γc^{-/-}) mice (Rongvaux et al, 2011). Unconditioned steady-state hTPOtg-RAG2^{-/-}γc^{-/-} mice were infused i.v. with 10⁷ GFP-ffLuc tagged control or c-MPL+ T cells in the retroorbital venous plexus for two consecutive doses, 6 days apart, followed by in vivo bioluminescent imaging (BLI) (FIGS. 1E-1F). C-MPL+ T cells had increased systemic persistence compared to control T cells (GFP-ffLuc+(n=10) vs GFP-ffLuc+c-MPL+(n=8), p=0.0003, t-test on log AUC for second T cell infusion), suggesting that c-MPL+ T cells received in vivo homeostatic cytokine signals in the presence of steady-state low systemic TPO levels (FIGS. 1G-1H). Human T cell persistence was detected in peripheral blood of 2/10 control mice and in 8/8 c-MPL+ T cell infused mice on days 15-17 (FIGS. 9A-9B).

### Example 2

### C-MPL in Tumor-Targeted TCR-Transgenic T Cells Provides Agonist-Dependent Enhancement of Anti-Tumor Function In Vitro

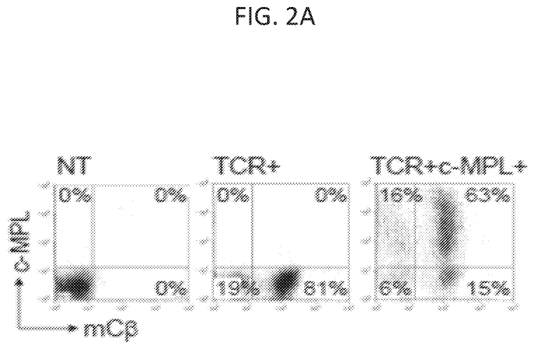

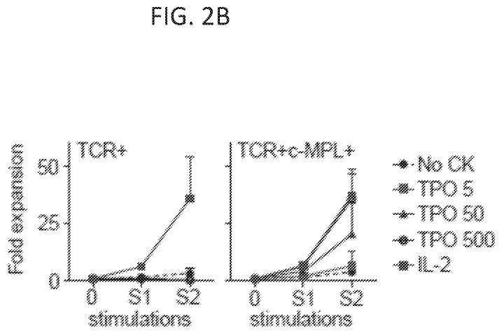

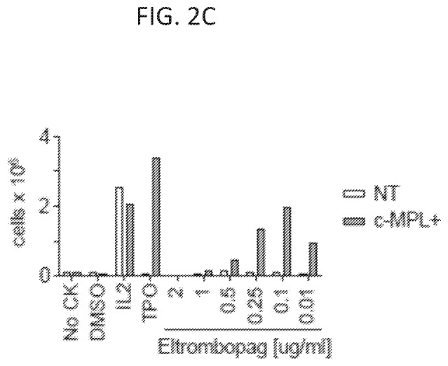

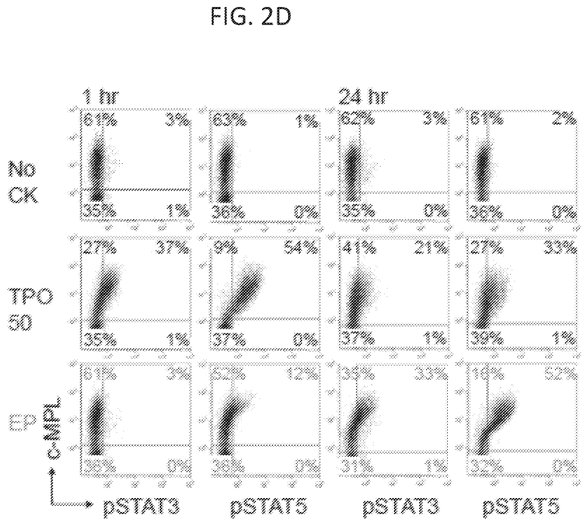

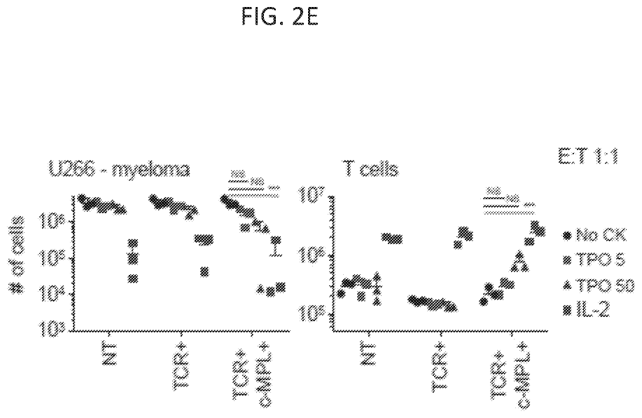

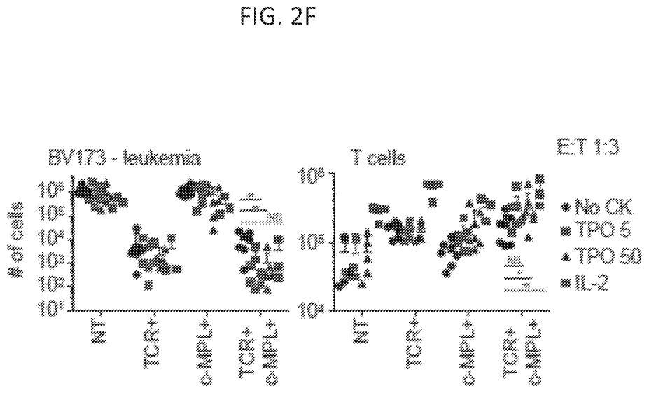

Previously described survivin-specific TCR-transgenic T cells were modified with c-MPL to assess agonist-dependent T cell expansion and enhanced anti-tumor function (Arber et al., 2015). CD8+ selected activated T cells were left non-transduced (NT), single transduced with the survivin-TCR (FIG. 7B), or co-transduced with the survivin-TCR and c-MPL vectors (FIGS. 7A-7B). Transductions were highly efficient (FIG. 2A), resulting in 58.9±12.6% TCR+c-MPL+, 19.1±9.7% TCR+c-MPL− and 12.1±5.5% TCR-c-MPL+ cells in the co-transduced group and 86.1±6.3% TCR+ cells in the TCR+ group (mean±SD, n=13, FIG. 2A). Next, it was tested if rhTPO supports antigen-specific T cell expansion of TCR+c-MPL+ T cells by stimulating TCR+ or TCR+c-MPL+ T cells with irradiated survivin peptide-pulsed artificial antigen presenting cells (aAPCs) in the presence of increasing concentrations of rhTPO (FIG. 2B). TCR+ T cells expanded in the presence of IL-2 but not in rhTPO, even in very high concentrations (500 ng/ml), or in the absence of cytokines (FIG. 2B). In contrast, TCR+c-MPL+ T cells readily expanded in a TPO-dose dependent manner (FIG. 2B) with comparable expansion levels to cells cultured in IL-2.

Similar results were obtained with the TPO small molecule agonist EP. A dose-titration experiment was used to determine the optimal concentration of EP to support c-MPL+ T cell expansion (FIG. 2C). EP was previously shown to inhibit leukemia cell proliferation at higher concentrations (1-10 μg/ml) by reduction of intracellular iron, a c-MPL independent effect (Roth et al., 2012). NT or c-MPL+ T cells were re-activated on OKT3/CD28-coated plates in the presence of different EP concentrations and expansion of viable T cells was determined after 7 days. An EP concentration of 0.1 μg/ml was retained for further in vitro studies as higher levels were toxic to the T cells and lower levels did not show c-MPL-dependent effects. Thus, the effective EP concentration for c-MPL+ T cells was about 1 log lower than in previous reports assessing effects of EP on megakaryocytic progenitor cells or leukemia cells *Roth et al., 2012; Will et al., 2009). To assess c-MPL ligand-dependent signaling in TCR+c-MPL+ T cells, cells were left untreated (noCK) or treated with TPO50 or EP for 1 (left) or 24 hours (right). Only c-MPL+ cells phosphorylated STAT3 and STATS upon treatment with rhTPO or EP, while c-MPL-cells did not, and EP treatment mostly led to JAK/STAT activation in c-MPL high expressing cells (FIG. 2D). Next, it was evaluated whether rhTPO enhanced the anti-tumor function of TCR+c-MPL+ T cells using two different HLA-A*0201+survivin+hematologic malignancies as targets (U266 multiple myeloma, BV173 leukemia) (FIGS. 2E-2F). Addition of rhTPO at 50 ng/ml to the co-cultures showed a trend toward enhanced killing of U266 cells (left) and enhanced persistence of TCR+c-MPL+ T cells (right) compared to no cytokine controls (FIG. 2E). Addition of IL2 25 U/ml however led to substantial enhancement of antigen independent tumor cell killing and T cell expansion (U266 with TCR+c-MPL+ T cells: noCK vs TPO50 43.3±1.1×10⁵ vs 22.8±11.2×10⁵, p=0.17; noCK vs IL2 43.3±1.1×10⁵ vs 3.13±0.45×10⁵, p<0.0001; T cell count: noCK vs TPO50 1.84±0.31×10⁵ vs 4.41±5.11×10⁵, p=0.59; noCK vs IL2 1.84±0.31×10⁵ vs 25.2±7.2×10⁵, p=0.0002; n=3, mean±SD). There was significantly enhanced killing of tumor cells in the presence of rhTPO at 50 ng/ml in the BV173 leukemia co-cultures (FIG. 2F) (BV173 with TCR+c-MPL+ T cells: noCK vs TPO50 10.3±7.9×10³ vs 0.99±×1.710³, p=0.002; noCK vs IL2 10.3±7.9×10³ vs 3.52±5.27×10³, p=0.13; T cell count: noCK vs TPO50 16.7±8.1×10⁴ vs 31.5±19.1×10⁴, p=0.05; noCK vs IL2 16.7±8.1×10⁴ vs 56.2±25.2×10⁴, p=0.006; n=7 for no CK, TPO5 and TPO50, n=3 for IL2, mean±SD). Again, addition of IL2 led to significant but antigen-independent enhancement of T cell expansion.

### Example 3

### C-MPL Activation Enables Sequential Killing Activity and Expansion of Central Memory TCR+C-MPL+ T Cells

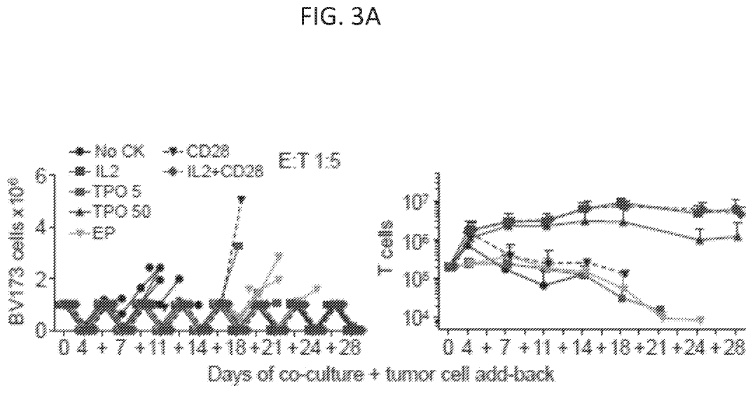

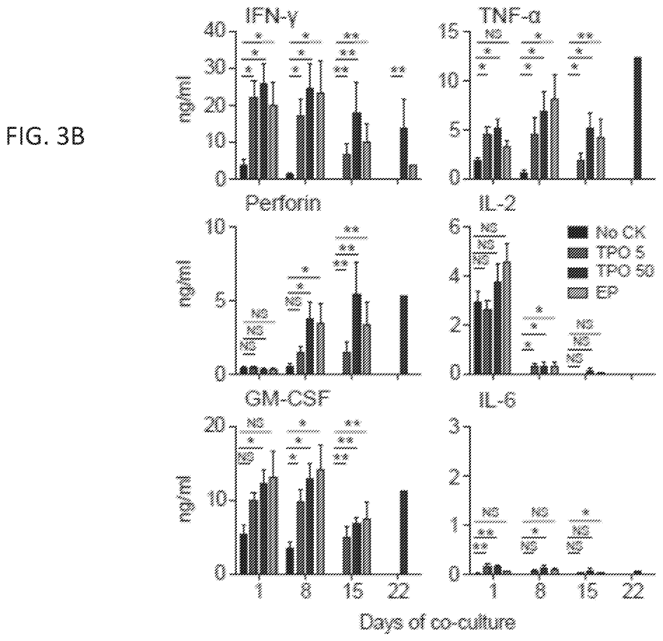

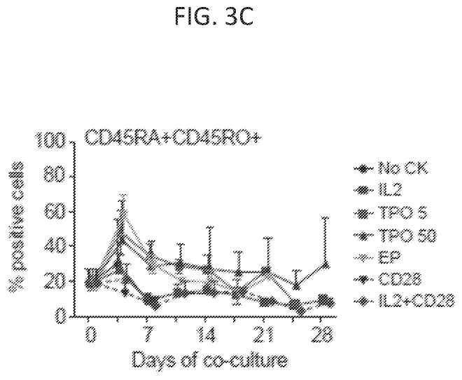

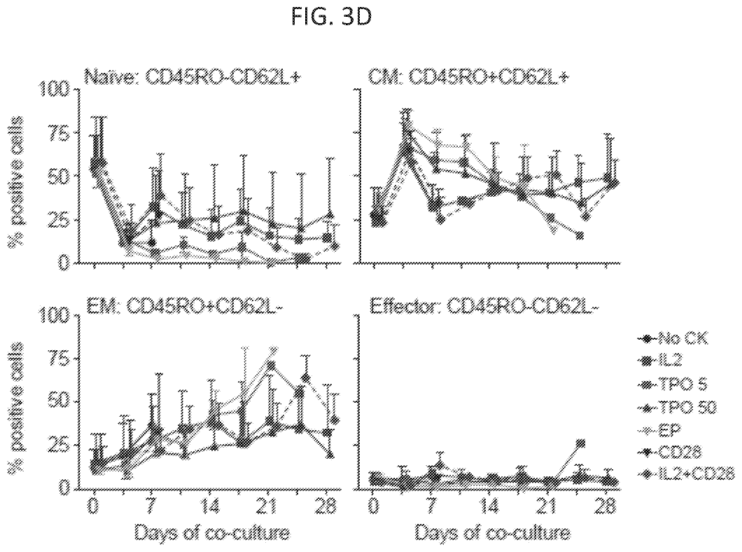

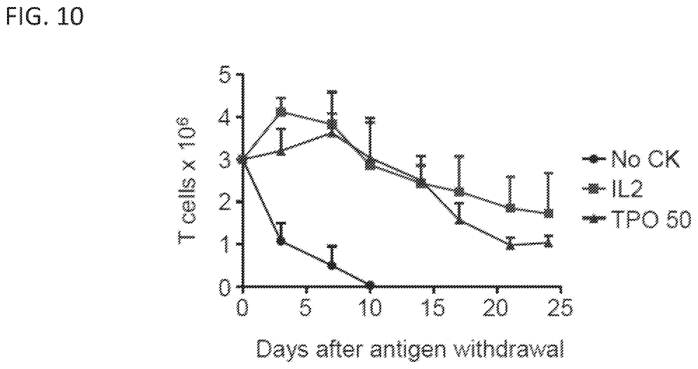

To study the effects of c-MPL signaling in TCR+ T cells in the presence of high tumor load, T cells were transduced with a polycistronic vector expressing both the TCR and c-MPL in a single construct (FIG. 7C). Viral copy number per cell was 4.87±2.48 copies (n=3, mean±SD) (FIG. 7D). TCR+c-MPL+ T cells were added to BV173 leukemia cells, and added these target cells back every 3-4 days to culture replicate wells up to eight times (FIG. 3A). TCR+c-MPL+ T cells in the absence of cytokines or with plate-bound CD28 alone killed only 1-2 times, while addition of rhTPO, EP, IL2 or plate-bound CD28+IL2 significantly increased the repetitive killing capacity of the cells up to 8 times (FIG. 3A, left), and also significantly enhanced T cell expansion (FIG. 3A, right). Time to cell killing was analyzed by Kaplan-Meier analysis and revealed consistent outcomes (overall p<0.0001). In addition, both rhTPO and EP sustained the levels of Th1 cytokine production in sequential co-cultures (FIG. 3B). Importantly, c-MPL stimulated sequential killer T cells did not show signs of growth factor independent T cell growth after withdrawal of antigen and c-MPL stimulation (FIG. 10). Persistent T cells were also analyzed for memory T cell markers over time. T cells co-cultured in rhTPO or EP showed enrichment in CD45RA+CD45RO+(FIG. 3C) and central memory cells (FIG. 3D) with lower proportions of effector memory cells. Naïve cells were enriched in some of the donors, but donor to donor variability was high (FIG. 3D).

### Example 4

### Immune Synapse Formation is More Efficient in T Cells with C-MPL Activation

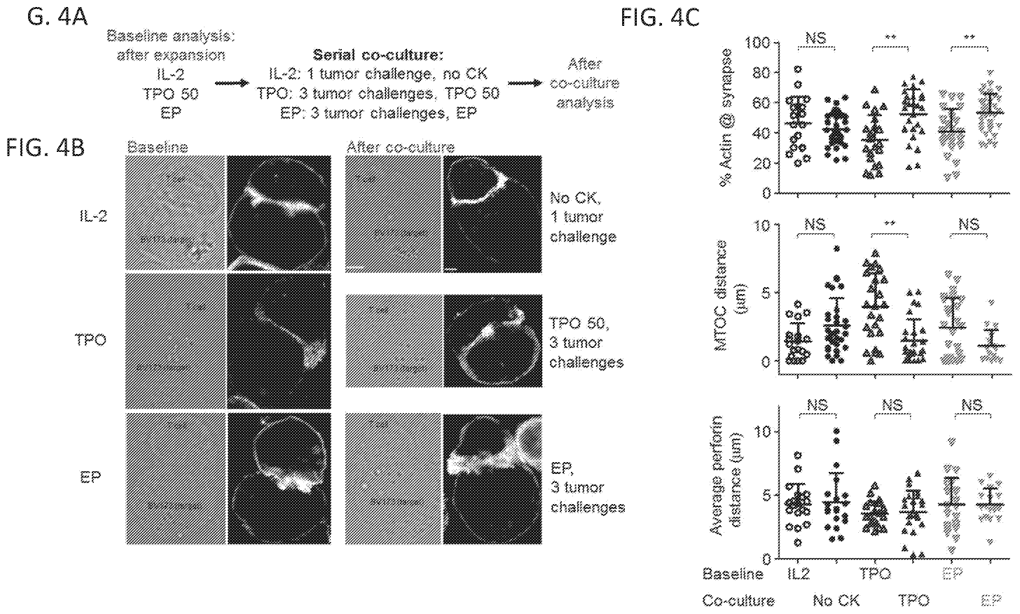

To analyze immune synapse formation between TCR+c-MPL+ T cells and BV173 leukemia target cells, confocal microscopy was performed and the inventors compared synapses formed under baseline conditions after T cell expansion to synapses formed from T cells purified after co-cultures (FIG. 4). In the absence of c-MPL activation, T cells were isolated after a single tumor cell challenge because these T cells do not kill repetitively and do not survive three tumor cell challenges. There was no detectable increased actin accumulation at the synapse (baseline vs co-culture 46.2±17.4% vs 42.2±10.9%, mean±SD, p=NS), shortening of the distance from the microtubule organization center to the synapse (MTOC distance, baseline vs co-culture 1.42±1.32 μm vs 2.57±2.02 μm, mean±SD, p=NS) or perforin convergence in TCR+c-MPL+ T cells re-isolated after one BV173 challenge (baseline vs co-culture 4.30±1.59 μm vs 4.47±2.27 μm, mean±SD, p=NS). To analyze the immune synapses formed by c-MPL stimulated sequential killer T cells, TCR+c-MPL+ T cells were re-isolated after three tumor cell challenges in the presence of rhTPO or EP. Both rhTPO or EP stimulated cells showed significantly increased actin accumulation at the synapse (TPO baseline vs co-culture 35.23±16.41% vs 52.60±16.32%, mean±SD, p=0.001; EP baseline vs co-culture 40.95±14.82% vs 53.29±12.47%, mean±SD, p=0.003) and shortening of the MTOC to synapse distance (TPO MTOC distance, baseline vs co-culture 3.96±2.49 μm vs 1.46±1.58 μm, mean±SD, p=0.01; EP MTOC distance, baseline vs co-culture 2.42±2.14 μm vs 1.09±1.16 μm, mean±SD, p=NS), indicating the formation of more efficient cytotoxic immune synapses in the presence of c-MPL signaling. Perforin convergence was unchanged regardless of c-MPL signaling (TPO baseline vs co-culture 3.53±1.00 μm vs 3.68±1.70 μm, mean±SD, p=NS; EP baseline vs co-culture 4.26±2.04 μm vs 4.25±1.28 μm, mean±SD, p=NS).

### Example 5

### C-MPL Signaling Produces Immune Stimulation in TCR-Transgenic Sequential Killer T Cells

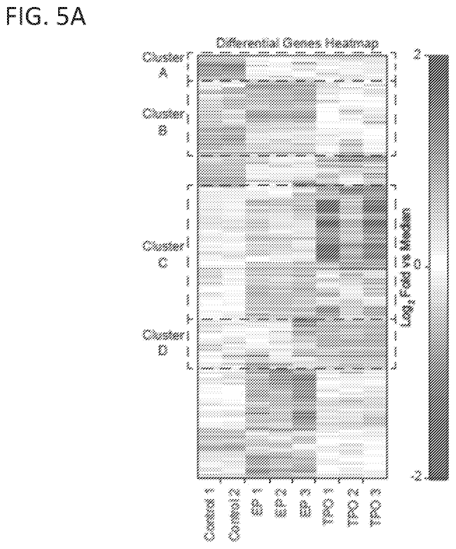

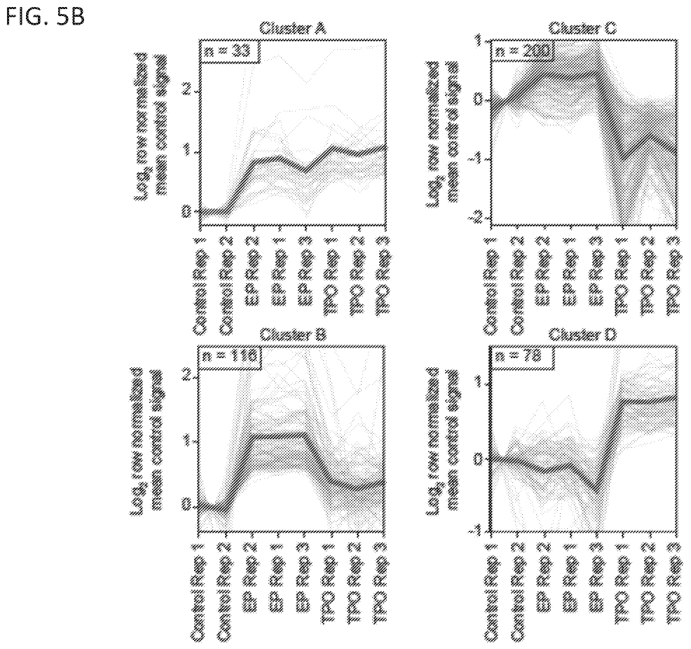

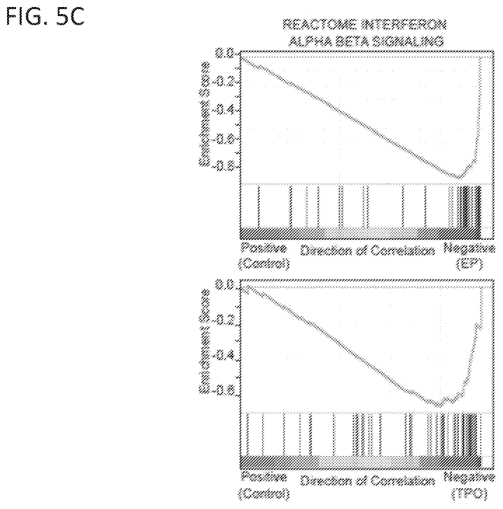

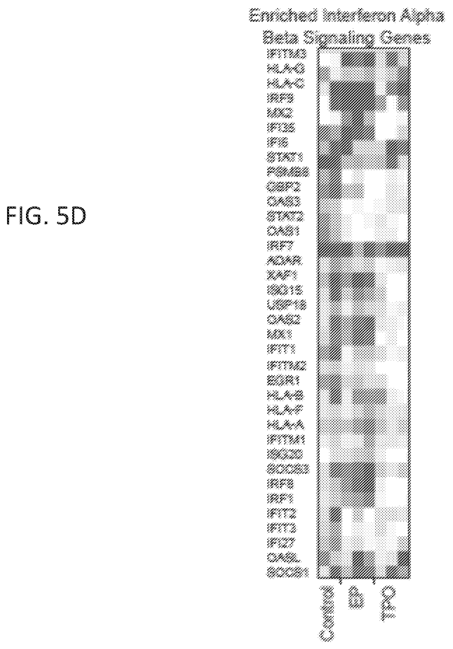

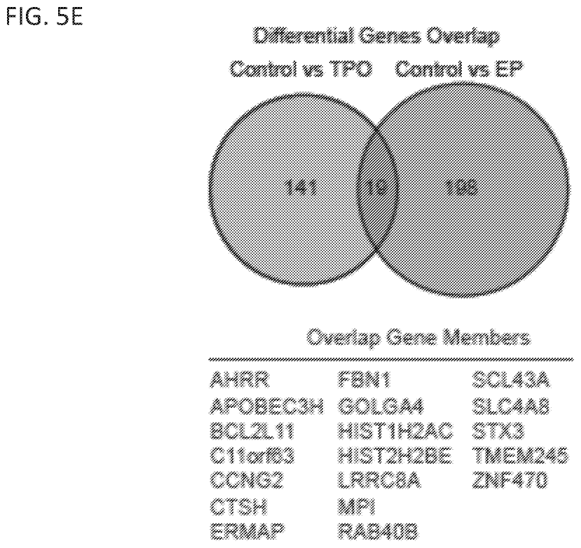

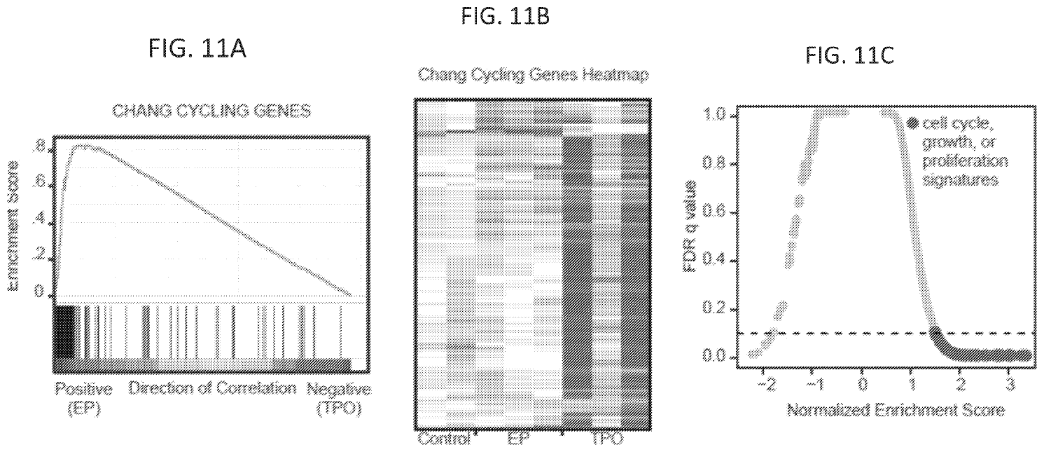

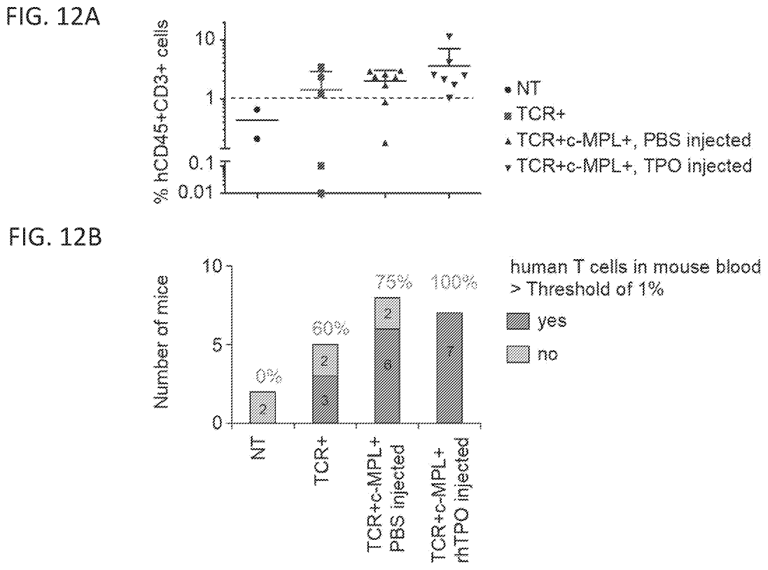

To study the molecular pathways affected by rhTPO- or EP-mediated c-MPL signaling in TCR-transgenic T cells during tumor cell killing, the gene expression profiles were analyzed of c-MPL enabled sequential killer T cells compared to TCR+c-MPL+ T cells in the absence of c-MPL activation (FIG. 5). Analogous to the experimental set-up of the synapse imaging studies, TCR+c-MPL+ T cells were subjected to multiple rounds of co-culture with BV173 leukemia cells, and RNA was isolated from purified surviving T cells after 1 tumor cell challenge for the control condition (co-culture in the absence of exogenous cytokines), or after 3 BV173 challenges in the presence of rhTPO or EP. The heatmap of differentially expressed genes led to the identification of two clusters with coherent upregulation of genes (clusters A and B) and two clusters with differentially regulated genes (clusters C and D) in co-cultured cells exposed to rhTPO or EP (FIGS. 5A-5B). Gene set enrichment analysis identified the IFN-α/β signaling pathway as the single most highly upregulated pathway in c-MPL stimulated co-cultures (FIGS. 5C-5D) and cell cycle associated genes as significantly differentially expressed in rhTPO vs EP treatment conditions (FIG. 11). Cell cycle associated genes were upregulated in T cells from EP-treated co-cultures versus controls and downregulated in T cells from rhTPO-treated co-cultures versus controls or EP-treatment, suggesting that during co-culture with tumor targets EP has a significant c-MPL independent effect on cell cycle genes (FIG. 11).

### Example 6

### C-MPL Signaling in TCR-Transgenic T Cells Enhances Anti-Tumor Function In Vivo

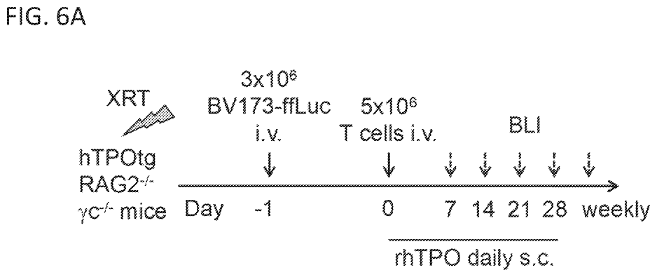

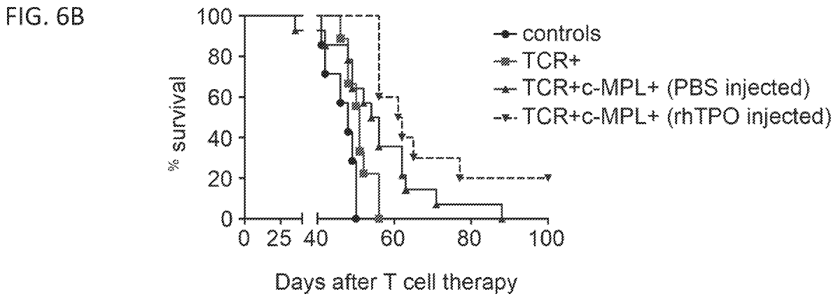

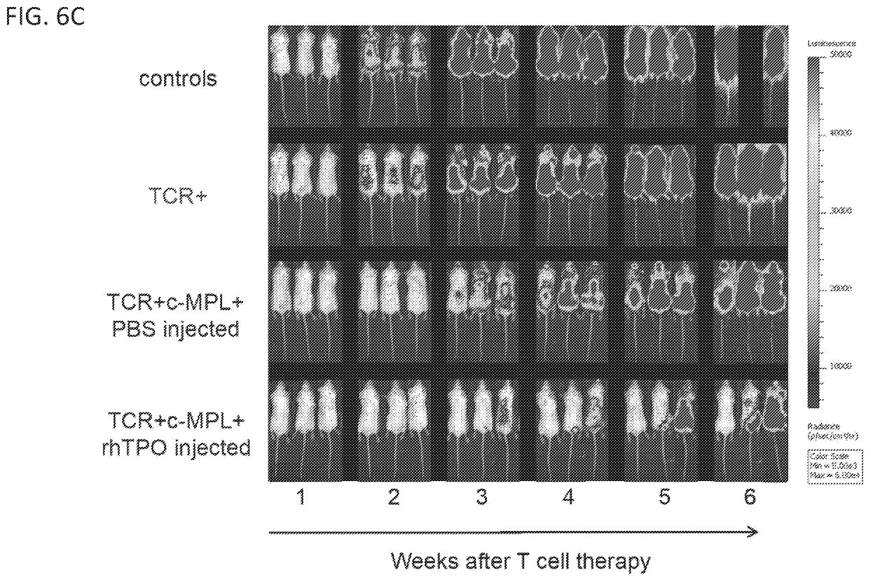

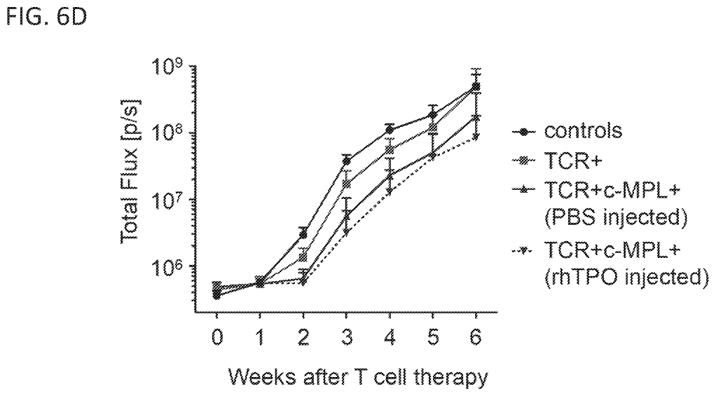

To test the in vivo anti-tumor function of TCR+c-MPL+ T cells in a mouse leukemia xenograft model, a previously described model (Arber et al., 2015) was adapted to hTPOtg-RAG2^{-/-}γc^{-/-} mice. To “stress” this system and better detect the effects of human TPO on TCR+c-MPL+ T cells, the same dose was given of 3×10⁶ BV173-ffLuc tumor cells to sublethally irradiated mice (Arber et al., 2015) but the administered T cell dose was reduced from 3 infusions of 10×10⁶ cells to a single infusion of 5×10⁶ cells, and omitted systemic T cell support with IL-2 injections post-infusion (FIG. 6A). To analyze homeostatic effects of TPO on transgenic T cells, mice receiving TCR+c-MPL+ T cells were either treated with daily s.c. saline (PBS) or with daily s.c. rhTPO (50 μg/kg/mouse) injections for the first 28 days. Groups of mice were compared to mice receiving TCR+ T cells alone, without c-MPL stimulation. It was tested if homeostatic TPO alone or the combination of homeostatic TPO and pharmacologic TPO treatment improved the anti-leukemic effect of TCR-transgenic T cells. Overall survival was significantly improved in the presence of c-MPL activation (p=0.004, FIG. 6B). Even with a single low dose of TCR+c-MPL+ T cells there was a trend to delayed leukemia growth by BLI in mice with steady-state TPO levels (p=NS) and a significant delay in mice with steady-state TPO levels and rhTPO injections (p=0.001) compared to TCR+ T cells alone (FIGS. 6C,6D). These results indicate a dose-response effect of c-MPL signaling in transgenic T cells when compared to mice receiving T cells without c-MPL stimulation (TCR+ group).

### Example 7

### Significance of Certain Embodiments

The present disclosure explored whether transgenic expression of the non-lymphoid hematopoietic growth factor receptor c-MPL in TCR-transgenic T cells benefits T cell survival and function following its activation. c-MPL signaling in T cells activates both co-stimulatory (signal 2) and cytokine receptor (signal 3) pathways in the presence of TCR signaling, thus leading to significantly enhanced anti-tumor function, immune synapse formation, cytokine production and T cell expansion/survival, all features that are typically suboptimal in TCR-transgenic T cells.

In polyclonal T cells, c-MPL activation leads to TPO-dependent T cell expansion and proliferation in vitro and to increased persistence in vivo in human TPO transgenic immunodeficient mice. These results illustrate that c-MPL signaling produces a homeostatic cytokine effect in T cells similar to common γ-chain cytokine signaling (such as IL-2), in the absence of TCR activation (signal 1). In addition, this effect is strictly dependent on the cognate ligand, since c-MPL+ T cells do not expand or proliferate in the absence of exogenously added rhTPO. Absence of cell autonomous ligand-independent growth supports the safety of the approach.

In tumor-targeted TCR-transgenic c-MPL+ T cells, c-MPL activation by either rhTPO or the small molecule agonist EP produces dose-dependent T cell expansion and enhances anti-tumor function. Indeed, c-MPL signaling enabled TCR+ T cells to sequentially kill tumor cells, and improved ligand-dependent Th1 cytokine production, preservation of a central memory phenotype, and had a potent effect on the formation of superior immune synapses. There was increased actin accumulation at the synapse as well as a better polarization of the MTOC to the synapse in the presence of c-MPL ligand. These components have been identified previously as essential indicators of effective lytic synapse formation in both native cytotoxic cells (Grakoui et al., 2015; Monks et al., 2015; McGavern et al., 2002) and engineered T cells (Hegde et al., 2016). Since efficient synapse formation also depends on the presence of several other receptors (e.g. adhesion molecules, co-stimulatory or checkpoint receptors) (Dustin et al., 2014), it was concluded that c-MPL activation during TCR stimulation and sequential tumor-cell killing provides additional signals required to produce stronger lytic synapses between engineered T cells and their target cells.

To further analyze the molecular events occurring in c-MPL+ TCR-transgenic cells during sequential cytotoxic activity, an unbiased RNA-seq analysis was performed. c-MPL stimulation upregulates genes in the type I interferon (IFN) pathway, providing potent immune-stimulatory signals to the engineered T cells, such as those seen during viral infections (Crouse et al., 2015). Previously, type I IFNs have been shown to potently support cytotoxic T cells by direct or indirect mechanisms during viral infection and also to enhance anti-cancer immunity (Zitvogel et al., 2015; Zhao et al., 2015). Additionally, type I IFNs increase the expression of perforin or granzyme B in cytotoxic T cells and promote the survival of memory T cells, and both were observed. These findings, together with the fact that c-MPL signaling activates multiple known cellular pathways that are shared by classical T cell co-stimulatory and cytokine receptors (Hitchcock and Kaushansky et al., 1994; Chen and Flies, 2013; Rochman et al., 2009) support the conclusion that c-MPL signaling in T cells can simultaneously produce both beneficial signals 2 and 3 in engineered T cells.

While many of the c-MPL-dependent gene expression changes were observed in both the TPO and EP treatment groups, there were significant differences between genes expressed in response to each stimulus. TPO-treated sequential killer T cells downregulated cell cycle, growth or proliferation signatures, while these pathways were upregulated in the EP-treated T cells. In specific embodiments, this can be attributed to EP treatment that can also have significant c-MPL independent effects on cells, as previously shown in acute myeloid leukemia cells (Roth et al., 2012; Sugita et al., 2013; Kalota et al., 2015). In gene expression studies performed on HL-60 AML cells treated with EP at 3 μg/ml, EP treatment led to down-regulation of cell cycle-associated genes with a block in the G1 phase of cell cycle (Roth et al., 2012). While EP treatment at 3 μg/ml was uniformly lethal to T cells, EP treatment at 0.1 μg/ml supported sequential killing by engineered T cells and significantly enhanced cell cycle and proliferation associated gene signatures. These results demonstrate the striking dose-dependent effects of EP on T cell cycle and proliferation pathways. In contrast, treatment of engineered T cells with rhTPO led to downregulation of cell cycle and proliferation pathways compared to controls or EP treated cells, consistent with the known ability of TPO-signaling in HSCs to induce quiescence and maintenance of the HSC pool (Hitchcock and Kaushansky, 2014).

TPO is not only produced systemically in the liver and kidneys, but also locally in the BM microenvironment by cells of the hematopoietic stem cell niche, such as stroma cells or osteoblasts, and also by malignant myeloid blasts (Yoshihara et al., 2007; Corazza et al., 2006). TPO is required for the maintenance of the HSC pool as it promotes HSC self-renewal and expansion in vivo, but can also induce HSC quiescence, a state critical to stem cell reservoir maintenance and avoiding premature exhaustion (Hitchcock and Kaushansky, 2014). In adoptively transferred T cells, a less differentiated phenotype is desirable, since these cells tend to persist longer in the host (Gattinoni et al., 2017; Sabatino et al., 2016; Biasco et al., 2015). In addition, TPO levels are significantly higher in BM than serum in steady-state and are substantially increased during chemotherapy-induced thrombocytopenia (Makar et al., 2013), in leukemic BM (Dong-Feng et al., 2014) and in a mouse model of myeloproliferative disease (Schepers et al., 2013). In that mouse model, elevated BM TPO levels contributed significantly to the re-modeling of a self-reinforcing leukemic stem cell niche that promoted progression of myeloproliferative disease (Schepers et al., 2013). C-MPL is therefore well suited for enhancing TCR-transgenic T cell activity against hematological malignancies, as there are at least five possible means by which benefit could be produced in vivo: (a) systemic low TPO serum levels mediate homeostatic cytokine signals, (b) local high TPO levels in the malignant BM microenvironment support local tumor-specific T cell expansion, (c) T cells with high c-MPL expression scavenge TPO from the tumor microenvironment and deprive leukemic blasts of the TPO signaling required for their survival, (d) administration of MPL-agonist drugs electively enhances transgenic T cell function, and (e) the window of post-chemotherapy thrombocytopenia accompanied by high serum TPO levels could be exploited for T cell infusion. In vivo in the human TPO-transgenic immunodeficient leukemia xenograft mouse model, steady-state TPO exerts a homeostatic cytokine effect on transferred c-MPL+TCR-transgenic T cells by slowing down leukemia progression. The combination of steady-state homeostatic TPO levels and pharmacologic dosing of rhTPO achieved the best result compared to mice receiving T cells in the absence of c-MPL activation (TCR+ group). The results provide a proof of concept that transgenic c-MPL in T cells can respond to either a soluble bone marrow microenvironment factor TPO or a receptor agonist drug (rhTPO).

Given the multiplicity of activities associated with forced expression of c-MPL, the approach can be used in a number of clinical settings. As specific cases, transgenic c-MPL+survivin-TCR+ T cells are valuable for survivin+HLA-A2+ myeloid malignancies (c-MPL+ T cells would lead to deprivation of TPO from the BM) and for survivin+HLA-A2+ lymphoid malignancies (c-MPL+ T cells benefit from endogenous TPO levels, c-MPL agonist drugs may be used to support transgenic T cell function).

In conclusion, this novel immuno-therapeutic strategy is useful to enhance the function and persistence of tumor-targeted TCR-engineered T cells with transgenic expression of the hematopoietic growth factor receptor c-MPL that can augment the anti-tumor activity of transgenic T cells by activation of both co-stimulatory and cytokine pathways, including type I IFN.

### Example 8

### Examples of Materials and Methods

Cell Lines.

BV173 cells (B cell acute lymphoblastic leukemia) were procured from the German Cell Culture Collection (DSMZ), the U266B1 (multiple myeloma) and K562 (erythroleukemia) cell lines from the American Type Culture Collection (ATCC), and maintained in RPMI media (Hyclone; Thermo Scientific) supplemented with 10 or 20% fetal bovine serum (FBS, Hyclone) according to manufacturer's recommendation, 1% penicillin-streptomycin (Gibco), and 1% glutamax (Gibco). 293T cells were obtained from the ATCC and maintained in complete IMDM media (Hyclone) (containing 10% FBS, 1% penicillin-streptomycin and 1% glutamax). For in vivo imaging experiments, the previously described BV173.ffLuc cell line was used (Arber et al. 2015). The K562 cell line was previously engineered to express the HLA-A*0201 molecule and CD40L, CD80, and OX40L as co-stimulatory molecules (Quintarelli et al., 2008) and used as artificial antigen presenting cells (aAPCs) for T cell expansion experiments. Cell lines were authenticated by the University of Texas MD Anderson Cancer Center Characterized Cell Line Core Facility and batches of cells were used for experiments within 6 months of authentication. Cell lines were also tested for mycoplasma contamination every 2 months.

Blood Samples from Healthy Donors.

Buffy coats were procured from de-identified healthy volunteers at the Gulf Coast Regional Blood Center (Houston, Tex., USA).

Generation of Retroviral Vectors.

The c-MPL plasmid was kindly provided by Dr. Patrick Barth, Baylor College of Medicine, Houston, USA, and the survivin-specific TCR has been described previously (Arber et al., 2015). Retroviral constructs (FIG. 7) were generated using the In-Fusion HD Cloning Kit (Clontech) according to manufacturer's instructions. Briefly, genes of interest were amplified by high-fidelity PCR, the SFG retroviral vector backbone linearized by restriction enzyme digest, bands of interest gel purified using the QIAquick Gel Extraction Kit (QIAGEN), appropriate fragments ligated into the retroviral backbone and transformed into stellar competent cells. Purified plasmid DNA was verified by sequencing (SeqWright or Epoch Life Science). The GFP-ffLuc retroviral vector producer cell line was kindly provided by Dr. Stephen Gottschalk, Baylor College of Medicine.

Generation of Retroviral Supernatant and Transduced T Cells.

Transient retroviral supernatant was prepared by transfection of 293T cells. Transient retroviral supernatant was prepared by transfection of 293T cells and activated T cells were transduced as described (Arber et al., 2015). The number of retroviral integrants in TCR+c-MPL+ T cells was estimated by quantitative PCR. Whole PBMC's were isolated from fresh buffy coats by Lymphoprep (Axis-Shield) density gradient centrifugation. If required, CD8+ T cells were isolated from PBMCs using magnetic beads and columns (Miltenyi Biotec). Cells were activated with plate-bound OKT3 (1 μg/μL), anti-CD28 antibodies (1 μg/μL) (BD) and IL2 (100 U/mL) and transduced 3 days later with retroviral supernatant on retronectin (Takara Bio) coated plates. T cells were collected after 48-72 hours and further expanded in CTL media (1:1 mixture of RPMI and Click's media, Hyclone) supplemented with 10% FBS, 1% penicillin-streptomycin, 1% glutamax, and either IL2 (50 U/mL), rhTPO (100 ng/mL) (Peprotech), or EP (0.1 μg/mL) (MedKoo). To enrich for c-MPL+ cells, a CD110+ selection was performed after labeling the cells with CD110-PE antibody (BD) followed by anti-PE magnetic beads (Miltenyi), for T cells expanded in media containing IL-2, before use in RNA sequencing, immunological synapse imaging, and in vivo experiments.

Real-Time Quantitative Polymerase Chain Reaction (Q-PCR) for TCR-c-MPL Transgene.

Genomic DNA extraction of T cells was performed using the QIAamp DNA Blood Mini Kit (Qiagen, Germantown, Md.), according to the manufacturer's instructions. Quantification of integrated transgene encoding TCR-c-MPL used real time Q-PCR with Custom TaqMan Gene Expression Assay (×20) and primers and probe designed to a specific sequence within the transgene (Fw Primer 5′GTCCAGCCAGTGTACTAAT-3′ (SEQ ID NO:3); Rv Primer 5′CTCAGGCCGAATTCCAT-3′(SEQ ID NO:4); Probe 5′ FAM-CTGGAGATGTTGAGAGCAATC-MGB-3′(SEQ ID NO:5)) and TaqMan Universal PCR Master Mix (×2) (both Applied Biosystems, Life Technologies by Thermo Fisher Scientific, Grand Island, N.Y.). Each Q-PCR reaction used 1000 ng of DNA in 25 uL reaction volume in an ABI 7900HT Sequence Detection System (Applied Biosystems, Grand Island, N.Y.) according to the manufacturer's instructions. Each sample was analyzed in triplicate. For the standard curve, serial dilutions were used of the plasmid encoding the transgene (from 300,000 to 3 copies per reaction). Copy number per diploid genome was calculated based on estimation that 1000 ng of genomic DNA contains 150,000 diploid human genomes as previously described (Di Stasi et al., 2011). Calculated copy number per diploid genome was normalized by transduction efficiency.

Immunophenotyping.

Cells were stained ith antibodies and analyzed by flow cytometry. Cells were stained with FITC-, phycoerythrin- (PE-), peridinin chlorophyll protein-(PerCP-), or allophycocyanin-conjugated (APC-conjugated) antibodies (Abs) against CD3, CD4, CD8, CD110 (c-MPL), murine TCR constant region (mCβ, ebioscience), CD19, or CD138. Cell viability was assessed by 7-AAD staining. T cells and tumor cell populations from co-culture assays were quantified by adding CountBright Beads (Invitrogen) to the analysis. T cell memory marker expression on CD8+ T cells re-covered from co-cultures was analyzed by staining cells with FITC-, PE-, ECD-, PerCP-, APC-, APC-AF750, or V450-conjugated Abs against CD45RO, CD45RA, CD62L, CCR7, CD110, mCβ, CD19, and 7AAD. Intracellular staining for phosphoproteins pSTAT3 and pSTAT5 was performed with PE- and Pacific Blue-conjugated Abs and Phosflow reagents (BD). To assess cell proliferation, cells were labeled with Carboxyfluorescein succinimidyl ester (CFSE, ThermoFisher) and analyzed after 7 days of culture. All antibodies were purchased from BD or Beckman Coulter unless otherwise indicated, and all staining procedures were performed according to the manufacturer's recommendation. Data acquisition was performed on a BD FACSCalibur using CellQuest software or a Beckman Coulter Gallios flow cytometer using Kaluza software. Data analysis was performed with FlowJo software (Tree Star Inc.).

Antigen-Specific Stimulation of T Cells and T Cell Expansions.

aAPCs (described above) were pulsed with the LMLGEFLKL survivin peptide (Genemed Synthesis), irradiated at 100 cGy, and used to stimulate T cells at an E:T ratio of 4:1 with IL-2 (50 U/mL) or rhTPO (5 ng/mL, 50 ng/mL, or 500 ng/mL). At the end of the first stimulation (7 days), cells were collected, counted, phenotyped and re-stimulated with fresh peptide-pulsed aAPCs under the same conditions. Fold expansion was recorded at the end of the second stimulation.

Co-Culture and Sequential Co-Culture Assay.

BV173 or U266B1 cells and T cells were co-cultured in up to eight replicate wells at various E:T ratios in CTL media with no cytokines, rhTPO (5 ng/mL or 50 ng/mL), or EP (0.1 μg/mL). Control conditions included IL2 (25 U/ml), anti-CD28 (1 μg/μL) (BD) coated plates or anti-CD28 coated plates and IL2 (25 U/ml). Co-culture supernatants were harvested 24 hours after initial plating or tumor-cell add-back to replicate wells and stored at −80° C. for further analysis. Every 3-4 days of co-culture, cells were harvested from a replicate culture well and analyzed by FACS for tumor- and T cell counts, as well as T cell memory marker phenotype. Fresh tumor cells and IL2, rhTPO or EP were added back at the same concentration as used for initial plating to untouched replicate wells. Co-cultures on anti-CD28 coated plates were transferred to new plates at each time-point.

Multiplex Cytokine Assay.

Co-culture supernatants were collected from the serial co-culture as described above. Co-culture supernatants were analyzed by the MILLIPLEX Human Th17 or Human CD8+ T-cell Magnetic Bead Panel (EMD Millipore) and Luminex 200 instrument (Luminex). Concentrations of IFN-γ, TNF-α, perforin, IL-2, GM-CSF, and IL-6 were determined in duplicates using the MILLIPLEX Human Th17 or the MILLIPLEX Human CD8+ T-cell Magnetic Bead Panel (EMD Millipore) and Luminex 200 instrument (Luminex) according to the manufacturer's instructions with one minor modification (assay buffer was used as the matrix solution instead of CTL media).

Immunological Synapse Imaging.

T cells isolated from sequential killing co-cultures and fresh BV173 target cells were mixed at E:T 1:2, incubated for 10 minutes, fixed, permeabilized, stained with the appropriate antibodies, and analyzed on a Leica TCS SP8 laser scanning microscope. T cells were co-cultured with BV173 cells at an E:T ratio of 1:5 in CTL media with no cytokines, rhTPO (50 ng/mL) or EP (0.1 μg/mL). After one killing (no cytokine condition) or three sequential killings (rhTPO and EP conditions), cells were collected and dead cells removed (Dead Cell Removal Kit, Miltenyi) (FIG. 4A). For confocal microscopy, T cells and fresh BV173 target cells were mixed at E:T ratio of 1:2, incubated for ten minutes in a tube and ten minutes on silane-coated glass slides (Electron Microscopy Sciences) at 37° C. Cells were then permeabilized and fixed with Perm/Fix solution (BD) for 15 minutes at room temperature and stained with anti-Pericentrin (rabbit, Abcam) and anti-Perforin Alexa-488 (mouse, BD Biosciences) primary antibodies. Phalloidin 568 (Life Technologies) was used to detect F-actin. Cells were imaged as Z stacks of 0.2 μm thickness to cover their entire volume on a Leica TCS SP8 laser scanning microscope using a 100× objective. Images were acquired with the LASAF software (Leica) and analyzed with Imaris software (Imaris) and Fiji.

RNA-Sequencing.

Total RNA was extracted from T cells of 3 independent donors, purified after sequential co-culture in no cytokine, rhTPO or EP (see above and FIG. 4A) using the RNeasy Mini Kit (Qiagen). T cell purification consisted of either dead cell removal alone (see above) or dead cell removal followed by FACS sorting (FACS Aria, BD, Flow Cytometry Core Laboratory at the Texas Children's Hospital Feigin Center), to achieve a >98% T cell purity and T cell viability of >90%. Total RNA samples were sent to GENEWIZ for library preparation and next generation sequencing, with 30 million reads per sample at a 1×50 base pair configuration. Data analysis is described in detail below.

RNA-Seq Data Analysis.

Code Availability.

Computational code used in analysis can be obtained at the following repository: https://github.com/linlabbcm/arber_ep_tpo.git

Genomic Coordinates and Gene Annotation.

All coordinates and gene annotations in this study were based on human reference genome assembly hg19 (ncbi.nlm.nih.gov/assembly/2758/) and RefSeq genes.

Aligning RNA-Seq Data and Analysis.

RNA-Seq was performed in triplicates from 3 independent donors for all 3 conditions (control, rhTPO and EP; see main text). Fastq files were aligned to the transcriptome using HiSat2 with default parameters (Kim et al., 2015). Following alignment and quality control analysis, one replicate of the control dataset was removed due to poor signal to noise ratio. Transcripts were assembled and FPKM values were generated using cuffquant and cuffnorm from the cufflinks software suite (Trapnell et al., 2010) Active transcripts were defined as transcripts with a normalized FPKM value greater than 1 in at least one sample.

Defining Differential Gene Expression.

Differential genes, defined as active genes with a log₂ fold change of at least +/−0.5849625 (equivalent to 1.5 fold in either direction) and an adjusted P-value less than or equal to 0.01 across comparisons of Control vs. EP, Control vs. TPO, and EP vs. TPO. In total 648 differential expressed genes were defined for subsequent analysis.

Creating Heat Map of Differential Gene Expression.

Heat maps were created for all differential genes clustered using hierarchical clustering. Each row plots the expression of active genes across Control reps, EP reps, and TPO Reps. Color intensities reflect the log 2 row mean normalized expression for each gene and bounded from −2 to 2 (FIG. 5A).

Identification of Gene Clusters with Similar Patterns of Expression.

Based on visual inspection of the heat map there were ˜10 clusters with distinct gene expression patterns. To define these in the data, the hierarchical cluster tree was cut to produce 10 distinct clusters. In particular, isolated clusters were identified for varied behaviors in EP response versus TPO response. Cluster A was chosen to show genes upregulated under both EP and TPO treatment, where TPO showed slightly stronger upregulation. Cluster B was chosen to show strong upregulation in both, where EP showed stronger upregulation. Cluster C was chosen to show upregulation in EP and downregulation in TPO. Finally, Cluster D was chosen to show downregulation under EP treatment and upregulation under TPO treatment. Clusters E-J have more varied expression patterns and were not included in subsequent analysis.

GSEA Data Analysis.

Gene Set Enrichment Analysis (GSEA) was used to identify gene sets correlating to the expression data found in the RNA-Seq data analysis. Gene sets were identified utilizing the outputs from the Control vs. EP, Control vs. TPO, and EP vs. TPO cufflinks analysis. GSEA was run with 1000 permutations, on the c2.all curated gene_set, where collapse dataset to gene symbols was set to false. The permutation type was gene_set, and the Chip platform used was GENE_SYMBOL.chip. The enrichment statistic used was weighted, and the metric for ranking genes was the log₂ ratio of classes. All other settings were default.

NES Vs FDR Gene Set Analysis.

NES vs FDR values from the output of the GSEA analysis were plotted, where cell cycle, growth, and proliferation gene sets highlighted in red were subjected to a<0.1 FDR cutoff.

Mouse Xenograft Models.

First Model: T Cell Persistence (FIGS. 1E-1H).

Female hTPOtg-RAG2^{-/-}γc⁻⁻ mice (stock #014594) were purchased at 4-8 weeks of age from the Jackson Laboratory and housed at the Baylor College of Medicine Animal Facility. Unirradiated steady-state mice were injected with two doses of 10⁷ control T cells or c-MPL+ T cells/mouse (both labeled with GFP-ffLuc), 6 days apart. T cell persistence was followed by in vivo bioluminescent imaging (BLI) (Caliper Life Sciences) and FACS analysis of peripheral blood of mice.

Second Model: Anti-Tumor Activity (FIG. 6).

Female hTPOtg-RAG2^{-/-}γc^{-/-} mice were irradiated with 200 cGy and injected with 3×10⁶ BV173.ffluc cells through the tail vein four to six hours later. The following day, 5×10⁶ T cells/mouse were injected through the retroorbital vein plexus. PBS or rhTPO (50 μg/kg/mouse) was injected subcutaneously daily for four weeks as indicated. Leukemia-growth was tracked over time by BLI. Sick mice were sacrificed, and organs (spleen, blood, BM, lymph nodes, and liver) were analyzed by FACS for the presence of leukemia and T cells.

Statistics.

Data were summarized using descriptive statistics. Areas under the curves (AUCs) were calculated using trapezoidal rule for T-cell frequencies and bioluminescent intensity over time. Comparisons were made between groups using Wilcoxon rank-sum test or t-test, whichever is appropriate, for continuous variables. Normality assumption was examined and log transformation was performed if necessary to achieve normality. Survival analysis was carried out using the Kaplan-Meier method. The Wilcoxon test was used to assess statistically significant differences between groups of mice. GraphPad Prism 5 software (GraphPad software, Inc., La Jolla, Calif.), SAS 9.4 and R 3.3.2 were used for statistical analysis. P values <0.05 were considered statistically significant.

Study Approval.

All animal studies were reviewed and approved by the IACUC of Baylor College of Medicine.

SEQUENCES

SEQ ID NO: 1

| | |
|---|---|
| 1 | cctgaaggga ggatgggcta aggcaggcac acagtggcgg agaagatgcc ctcctgggcc |
| 61 | ctcttcatgg tcacctcctg cctcctcctg gcccctcaaa acctggccca agtcagcagc |
| 121 | caagatgtct ccttgctggc atcagactca gagcccctga agtgtttctc ccgaacattt |
| 181 | gaggacctca cttgcttctg ggatgaggaa gaggcagcgc ccagtgggac ataccagctg |
| 241 | ctgtatgcct acccgcggga gaagccccgt gcttgccccc tgagttccca gagcatgccc |
| 301 | cactttggaa cccgatacgt gtgccagttt ccagaccagg aggaagtgcg tctcttcttt |
| 361 | ccgctgcacc tctgggtgaa gaatgtgttc ctaaaccaga ctcggactca gcgagtcctc |
| 421 | tttgtggaca gtgtaggcct gccggctccc cccagtatca tcaaggccat gggtgggagc |
| 481 | cagccagggg aacttcagat cagctgggag gagccagctc cagaaatcag tgatttcctg |
| 541 | aggtacgaac tccgctatgg ccccagagat cccaagaact ccactggtcc cacggtcata |
| 601 | cagctgattg ccacagaaac ctgctgccct gctctgcaga ggcctcactc agcctctgct |
| 661 | ctggaccagt ctccatgtgc tcagcccaca atgccctggc aagatggacc aaagcagacc |
| 721 | tccccaagta gagaagcttc agctctgaca gcagagggtg gaagctgcct catctcagga |
| 781 | ctccagcctg gcaactccta ctggctgcag ctgcgcagcg aacctgatgg gatctccctc |
| 841 | ggtggctcct ggggatcctg gtccctccct gtgactgtgg acctgcctgg agatgcagtg |
| 901 | gcacttggac tgcaatgctt taccttggac ctgaagaatg ttacctgtca atggcagcaa |
| 961 | caggaccatg ctagctccca aggcttcttc taccacagca gggcacggtg ctgccccaga |
| 1021 | gacaggtacc ccatctggga gaactgcgaa gaggaagaga aaacaaatcc aggactacag |
| 1081 | accccacagt tctctcgctg ccacttcaag tcacgaaatg acagcattat tcacatcctt |
| 1141 | gtggaggtga ccacagcccc gggtactgtt cacagctacc tgggctcccc tttctggatc |
| 1201 | caccaggctg tgcgcctccc caccccaaac ttgcactgga gggagatctc cagtgggcat |
| 1261 | ctggaattgg agtggcagca cccatcgtcc tgggcagccc aagagacctg ttatcaactc |
| 1321 | cgatacacag gagaaggcca tcaggactgg aaggtgctgg agccgcctct cggggcccga |
| 1381 | ggagggaccc tggagctgcg cccgcgatct cgctaccgtt tacagctgcg cgccaggctc |
| 1441 | aacggcccca cctaccaagg tccctggagc tcgtggtcgg acccaactag ggtggagacc |
| 1501 | gccaccgaga ccgcctggat ctccttggtg accgctctgc atctagtgct gggcctcagc |
| 1561 | gccgtcctgg gcctgctgct gctgaggtgg cagtttcctg cacactacag gagactgagg |
| 1621 | catgccctgt ggccctcact tccagacctg caccgggtcc taggccagta ccttagggac |
| 1681 | actgcagccc tgagcccgcc caaggccaca gtctcagata cctgtgaaga agtggaaccc |
| 1741 | agcctccttg aaatcctccc caagtcctca gagaggactc ctttgcccct gtgttcctcc |
| 1801 | caggcccaga tggactaccg aagattgcag ccttcttgcc tggggaccat gcccctgtct |
| 1861 | gtgtgcccac ccatggctga gtcagggtcc tgctgtacca cccacattgc caaccattcc |
| 1921 | tacctaccac taagctattg gcagcagcct tgaggacagg ctcctcactc ccagttccct |
| 1981 | ggacagagct aaactctcga gacttctctg tgaacttccc taccctaccc ccacaacaca |
| 2041 | agcaccccag acctcacctc catccccctc tgtctgccct cacaattagg cttcattgca |
| 2101 | ctgatcttac tctactgctg ctgacataaa accaggaccc tttctccaca ggcaggctca |
| 2161 | tttcactaag ctcctccttt actttctctc tcctctttga tgtcaaacgc cttgaaaaca |
| 2221 | agcctccact tccccacact tcccatttac tcttgagact acttcaatta gttcccctac |
| 2281 | tacactttgc tagtgaaact gcccaggcaa agtgcacctc aaatcttcta attccaagat |
| 2341 | ccaataggat ctcgttaatc atcagttcct ttgatctcgc tgtaagattt gtcaaggctg |
| 2401 | actactcact tctcctttaa attctttcct accttggtcc tgcctctttg agtatattag |
| 2461 | taggtttttt ttatttgttt gagacagggt ctcactctgt cacccaggct gcagtgcaat |
| 2521 | ggcgcgatct cagctcactg caacctccac ctccgggttc aagcgattct tgtgcctcgg |
| 2581 | cctccctagt agctgggatt acaggcgcac accaccacac acagctaatt tttttttttt |
| 2641 | tttttttttt ttttttttag acggagcctt gctctgttgc cagactggag tgcagtggca |
| 2701 | cgatctcggc tcactgcaac ctctgcctcc cgggttcaag ccattctgcc tcagcctccc |
| 2761 | aagtagctgg gagtacaggc gtctgccacc atgcctaatt tttttctatt tttaggagag |
| 2821 | accggttttc accacgttgg ccaggatggt ctcgatatcc tgatctcgtg atccgcctgc |
| 2881 | ctctgcctcc caaagtgctg ggattacagg tgtgacccac tgcgcacagc cccagctaat |
| 2941 | tttcatattt ttagtagaga cagggttttg ccatgttgcc caggctggtc ttgaactcct |
| 3001 | aacctcgggt gatccaccca ccttggcctc ccaaagtgtt aggattacag gcatgagcca |
| 3061 | ctgcgcccgg ctgagtgtac tagtagttaa gagaataaac tagatctaga atcagagctg |
| 3121 | gattcaattc ctgtccttca catttactag ctgtgcaacc ttgggcacat aacttaatgt |
| 3181 | ctttgagcct tagttttttc atctgtaaaa cagggataat aacagcaccc catagagttg |
| 3241 | tgacgaggat tgagataatc taagtaaagc acagtcccta ggacatagta aatgattcat |
| 3301 | atatccgaac tactgttata attattcctt cttactctcc tcttctagca tttcttccaa |
| 3361 | ttattacagt ccttcaagat tccatttctt aacagtctcc aatcccatct attctctgcc |
| 3421 | tttactatat gttgaccatt ccaaagttct tatctctagc tcagacatct actacagcac |
| 3481 | tgtgatgctt tatgcaacta actgtttaca tatctgtccc ctgctactag attgtgagct |
| 3541 | ccttgaggga aaggaacatg atttatttgt ccttttcccc cagcacctag agtagtgctt |
| 3601 | ggtgcatgat agtaggcctt caataaattt tttctaaatg aatga |

SEQ ID NO: 2
MPSWALFMVTSCLLLAPQNLAQVSSQDVSLLASDSEPLKCFSRT
FEDLTCFWDEEEAAPSGTYQLLYAYPREKPRACPLSSQSMPHFGTRYVCQFPDQEEVR
LFFPLHLWVKNVFLNQTRTQRVLFVDSVGLPAPPSIIKAMGGSQPGELQISWEEPAPE
ISDFLRYELRYGPRDPKNSTGPTVIQLIATETCCPALQRPHSASALDQSPCAQPTMPW
QDGPKQTSPSREASALTAEGGSCLISGLQPGNSYWLQLRSEPDGISLGGSWGSWSLPV
TVDLPGDAVALGLQCFTLDLKNVTCQWQQQDHASSQGFFYHSRARCCPRDRYPIWENC
EEEEKTNPGLQTPQFSRCHFKSRNDSIIHILVEVTTAPGTVHSYLGSPFWIHQAVRLP
TPNLHWREISSGHLELEWQHPSSWAAQETCYQLRYTGEGHQDWKVLEPPLGARGGTLE
LRPRSRYRLQLRARLNGPTYQGPWSSWSDPTRVETATETAWISLVTALHLVLGLSAVL
GLLLLRWQFPAHYRRLRHALWPSLPDLHRVLGQYLRDTAALSPPKATVSDTCEEVEPS
LLEILPKSSERTPLPLCSSQAQMDYRRLQPSCLGTMPLSVCPPMAESGSCCTTHIANH
SYLPLSYWQQP

### REFERENCES

All patents, patent applications, and publications cited herein are hereby incorporated by reference in their entirety herein.
Arber C, Feng X, Abhyankar H, Romero E, Wu M F, Heslop H E et al. Survivin-specific T cell receptor targets tumor but not T cells. J Clin Invest 2015; 125(1): 157-68.
Biasco L, Scala S, Basso Ricci L, Dionisio F, Baricordi C, Calabria A et al. In vivo tracking of T cells in humans unveils decade-long survival and activity of genetically modified T memory stem cells. Sci Transl Med 2015; 7(273): 273ra13.
Chang M, Suen Y, Meng G, Buzby J S, Bussel J, Shen V et al. Differential mechanisms in the regulation of endogenous levels of thrombopoietin and interleukin-11 during thrombocytopenia: insight into the regulation of platelet production. Blood 1996; 88(9): 3354-62.
Cheever M A, Allison J P, Ferris A S, Finn O J, Hastings B M, Hecht T T et al. The prioritization of cancer antigens: a national cancer institute pilot project for the acceleration of translational research. Clin Cancer Res 2009; 15(17): 5323-37.
Chen L, Flies D B. Molecular mechanisms of T cell co-stimulation and co-inhibition. Nat Rev Immunol 2013; 13(4): 227-42.
Corazza F, Hermans C, D'Hondt S, Ferster A, Kentos A, Benoit Y et al. Circulating thrombopoietin as an in vivo growth factor for blast cells in acute myeloid leukemia. Blood 2006; 107(6): 2525-30.
Crouse J, Kalinke U, Oxenius A. Regulation of antiviral T cell responses by type I interferons. Nat Rev Immunol 2015; 15(4): 231-42.
Di Stasi A, Tey S K, Dotti G, Fujita Y, Kennedy-Nasser A, Martinez C, Straathof K, Liu E, Durett A G, Grilley B, Liu H, Cruz C R, Savoldo B, Gee A P, Schindler J, Krance R A, Heslop H E, Spencer D M, Rooney C M, Brenner M K. Inducible apoptosis as a safety switch for adoptive cell therapy. NEJM 2011, 365(18): 1673-83.
Dong-Feng Z, Ting L, Yong Z, Cheng C, Xi Z, Pei-Yan K. The TPO/c-MPL Pathway in the Bone Marrow may Protect Leukemia Cells from Chemotherapy in AML Patients. Pathol Oncol Res 2014; 20(2): 309-17.
Dotti G, Gottschalk S, Savoldo B, Brenner M K. Design and development of therapies using chimeric antigen receptor-expressing T cells. Immunol Rev 2014; 257(1): 107-26.
Dustin M L. The immunological synapse. Cancer Immunol Res 2014; 2(11): 1023-33.
Fesnak A D, June C H, Levine B L. Engineered T cells: the promise and challenges of cancer immunotherapy. Nat Rev Cancer 2016; 16(9): 566-81.
Fox N, Priestley G, Papayannopoulou T, Kaushansky K. Thrombopoietin expands hematopoietic stem cells after transplantation. J Clin Invest 2002; 110(3): 389-94.
Gattinoni L, Speiser D E, Lichterfeld M, Bonini C. T memory stem cells in health and disease. Nat Med 2017; 23(1): 18-27.
Grakoui A, Bromley S K, Sumen C, Davis M M, Shaw A S, Allen P M et al. Pillars article: The immunological synapse: a molecular machine controlling T cell activation. Science. 1999. 285: 221-227. J Immunol 2015; 194(9): 4066-72.
Grozovsky R, Begonja A J, Liu K, Visner G, Hartwig J H, Falet H et al. The Ashwell-Morell receptor regulates hepatic thrombopoietin production via JAK2-STAT3 signaling. Nat Med 2015; 21(1): 47-54.
Hegde M, Mukherjee M, Grada Z, Pignata A, Landi D, Navai S A et al. Tandem CAR T cells targeting HER2 and IL13Ralpha2 mitigate tumor antigen escape. J Clin Invest 2016; 126(8): 3036-52.
Hitchcock I S, Kaushansky K. Thrombopoietin from beginning to end. Br J Haematol 2014; 165(2): 259-68.
Kalota A, Selak M A, Garcia-Cid L A, Carroll M. Eltrombopag modulates reactive oxygen species and decreases acute myeloid leukemia cell survival. PLoS One 2015; 10(4): e0126691.
Kaushansky K, Lok S, Holly R D, Broudy V C, Lin N, Bailey M C et al. Promotion of megakaryocyte progenitor expansion and differentiation by the c-Mpl ligand thrombopoietin. Nature 1994; 369(6481): 568-71.
Kershaw M H, Westwood J A, Darcy P K. Gene-engineered T cells for cancer therapy. Nat Rev Cancer 2013; 13(8): 525-41.
Kim D, Langmead B, Salzberg S L. HISAT: a fast spliced aligner with low memory requirements. Nat Methods 2015; 12(4): 357-60.
Makar R S, Zhukov O S, Sahud M A, Kuter D J. Thrombopoietin levels in patients with disorders of platelet production: diagnostic potential and utility in predicting response to TPO receptor agonists. Am J Hematol 2013; 88(12): 1041-4.
Monks C R, Freiberg B A, Kupfer H, Sciaky N, Kupfer A. Pillars article: Three-dimensional segregation of supramolecular activation clusters in T cells. Nature. 1998. 395: 82-86. J Immunol 2015; 194(9): 4061-5.
McGavern D B, Christen U, Oldstone M B. Molecular anatomy of antigen-specific CD8(+) T cell engagement and synapse formation in vivo. Nat Immunol 2002; 3(10): 918-25.
Qian H, Buza-Vidas N, Hyland C D, Jensen C T, Antonchuk J, Mansson R et al. Critical role of thrombopoietin in maintaining adult quiescent hematopoietic stem cells. Cell Stem Cell 2007; 1(6): 671-84.
Quintarelli C, Dotti G, De Angelis B, Hoyos V, Mims M, Luciano L et al. Cytotoxic T lymphocytes directed to the preferentially expressed antigen of melanoma (PRAME) target chronic myeloid leukemia. Blood 2008; 112(5): 1876-85.
Rochman Y, Spolski R, Leonard W J. New insights into the regulation of T cells by gamma(c) family cytokines. Nat Rev Immunol 2009; 9(7): 480-90.
Rongvaux A, Willinger T, Takizawa H, Rathinam C, Auerbach W, Murphy A J et al. Human thrombopoietin knockin mice efficiently support human hematopoiesis in vivo. Proc Natl Acad Sci USA 2011; 108(6): 2378-83.
Roth M, Will B, Simkin G, Narayanagari S, Barreyro L, Bartholdy B et al. Eltrombopag inhibits the proliferation of leukemia cells via reduction of intracellular iron and induction of differentiation. Blood 2012; 120(2): 386-94.
Sabatino M, Hu J, Sommariva M, Gautam S, Fellowes V, Hocker J D et al. Generation of clinical-grade CD19-specific CAR-modified CD8+ memory stem cells for the treatment of human B-cell malignancies. Blood 2016; 128(4): 519-28.
Schepers K, Pietras E M, Reynaud D, Flach J, Binnewies M, Garg T et al. Myeloproliferative neoplasia remodels the endosteal bone marrow niche into a self-reinforcing leukemic niche. Cell Stem Cell 2013; 13(3): 285-99.
Sugita M, Kalota A, Gewirtz A M, Carroll M. Eltrombopag inhibition of acute myeloid leukemia cell survival does not depend on c-Mpl expression. Leukemia 2013; 27(5): 1207-10.
Trapnell C, Williams B A, Pertea G, Mortazavi A, Kwan G, van Baren M J et al. Transcript assembly and quantification by RNA-Seq reveals unannotated transcripts and isoform switching during cell differentiation. Nat Biotechnol 2010; 28(5): 511-5.
Will B, Kawahara M, Luciano J P, Bruns I, Parekh S, Erickson-Miller C L et al. Effect of the nonpeptide thrombopoietin receptor agonist Eltrombopag on bone marrow cells from patients with acute myeloid leukemia and myelodysplastic syndrome. Blood 2009; 114(18): 3899-908.
Yoshihara H, Arai F, Hosokawa K, Hagiwara T, Takubo K, Nakamura Y et al. Thrombopoietin/MPL signaling regulates hematopoietic stem cell quiescence and interaction with the osteoblastic niche. Cell Stem Cell 2007; 1(6): 685-97.
Zitvogel L, Galluzzi L, Kepp O, Smyth M J, Kroemer G. Type I interferons in anticancer immunity. Nat Rev Immunol 2015; 15(7): 405-14.
Zhao Z, Condomines M, van der Stegen S J, Perna F, Kloss C C, Gunset G et al. Structural Design of Engineered Co stimulation Determines Tumor Rejection Kinetics and Persistence of CAR T Cells. Cancer Cell 2015; 28(4): 415-28.

Although the present invention and its advantages have been described in detail, it should be understood that various changes, substitutions and alterations can be made herein without departing from the spirit and scope of the invention as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure of the present invention, processes, machines, manufacture, compositions of matter, means, methods, or steps, presently existing or later to be developed that perform substantially the same function or achieve substantially the same result as the corresponding embodiments described herein may be utilized according to the present invention. Accordingly, the appended claims are intended to include within their scope such processes, machines, manufacture, compositions of matter, means, methods, or steps.

1. An immune cell, comprising recombinant expression of the thrombopoietin receptor (hematopoietic growth factor receptor, c-MPL).

2. The cell of claim 1, wherein there is no expression of endogenous c-MPL in the cell or wherein an existing expression of c-MPL is overexpressed upon recombinant expression of c-MPL.

3. The cell of claim 1, wherein the immune cell is an alpha beta T cell, gamma delta T cell, NK cell, NKT cell, tumor infiltrating lymphocyte, or bone marrow infiltrating lymphocyte.

4. The cell of claim 1, wherein the immune cell comprises an engineered receptor.

5. The cell of claim 4, wherein the engineered receptor comprises a transgenic T cell receptor (TCR).

6. The cell of claim 4, wherein the engineered receptor comprises a chimeric antigen receptor (CAR).

7. The cell of claim 4, wherein the engineered receptor and/or an endogenous receptor targets a tumor-associated antigen.

8. The cell of claim 7, wherein the tumor-associated antigen is EphA2, HER2, GD2, Glypican-3, 5T4, 8H9, αᵥβ₆ integrin, B cell maturation antigen (BCMA) B7-H3, B7-H6, CAIX, CA9, CD19, CD20, CD22, kappa light chain, CD30, CD33, CD38, CD44, CD44v6, CD44v7/8, CD70, CD123, CD138, CD171, CS1, CSPG4, EGFR, EGFRvIII, EGP2, EGP40, EPCAM, ERBB3, ERBB4, ErbB3/4, FAP, FAR, FBP, fetal AchR, Folate Receptor α, GD3, HLA-AI, HLA-A2, IL11Ra, IL13Ra2, KDR, lambda light chain, Lewis-Y, MCSP, Mesothelin, Muc1, Muc16, NCAM, NKG2D ligands, NY-ESO-1, PRAME, PSCA, PSC1, PSMA, ROR1, Sp17, survivin, TAG72, TEM1, TEM8, carcinoembryonic antigen, HMW-MAA, VEGF receptors, MAGE-A1, MAGE-A3, MAGE-A4, CT83, SSX2, XIAP, cIAP1, cIAP2, NAIP, and/or Livin.

9. The cell of claim 8, wherein the tumor-associated antigen is survivin.

10. The cell of claim 1, wherein c-MPL is expressed via a recombinant expression vector operable in eukaryotic cells.

11. The cell of claim 1, wherein the expression of c-MPL is regulated by a constitutive promoter.

12. The cell of claim 1, wherein the expression of c-MPL is regulated by an inducible promoter.

13. The cell of claim 10, wherein the vector is a viral vector.

14. The cell of claim 13, wherein the viral vector is a retrovirus, lentivirus, adenovirus, adeno-associated virus, or herpes simplex virus.

15. The cell of claim 10, wherein the vector is a non-viral vector.

16. The cell of claim 15, wherein the non-viral vector is naked DNA or plasmid DNA or minicircle DNA.

17. The cell of claim 1, wherein the c-MPL is a functionally active fragment or variant of c-MPL.

18. A method of improving immune cell therapy, comprising the step of modifying the immune cells to express c-MPL or parts thereof.

19. The method of claim 18, wherein the cells comprise immune cells comprising recombinant expression of the thrombopoietin receptor (hematopoietic growth factor receptor, c-MPL).

20. The method of claim 18, wherein the cell therapy is for a malignancy in an individual.

21. The method of claim 20, wherein the malignancy comprises acute lymphoblastic leukemia, acute myelogenous leukemia, chronic lymphocytic leukemia, chronic myelogenous leukemia, acute monocytic leukemia, Hodgkin's lymphoma, non-Hodgkin's lymphoma, and/or solid tumors.

22. The method of claim 21, wherein the solid tumors comprise tumors of the brain, breast, bladder, bone, colon, rectum, cervix, endometrium, esophagus, eye, gallbladder, hypopharynx, kidney, larynx, liver, lung, nasopharynx, oropharynx, ovary, pancreas, penis, pituitary, prostate, skin, small intestine, stomach, testes, thymus, thyroid, uterus, vagina and/or vulva.

23. A method for improving immune cell persistence and/or function, comprising the step of activating immune cells that express recombinant c-MPL by subjecting the cells to thrombopoietin (TPO) and/or one or more agonists of c-MPL.

24. The method of claim 23, wherein the cells comprise recombinant expression of the thrombopoietin receptor (hematopoietic growth factor receptor, c-MPL).

25. The method of claim 23, wherein the activating step occurs ex vivo.

26. The method of claim 23, wherein the activating step occurs in vitro.

27. The method of claim 23, wherein the activating step occurs in vivo.

28. The method of claim 23, wherein the cells are exposed to TPO.

29. The method of claim 23, wherein the cells are exposed to one or more agonists of c-MPL.

30. The method of claim 29, wherein the agonist is eltrombopag (EP), NIP-004 or other small molecule agonists, romiplostim or other peptide agonists, or a combination thereof.

31. A method for treating cancer in an individual, comprising the step of delivering to the individual a therapeutically effective amount of immune cells of claim 1.

32. The method of claim 31, wherein the method further comprises the step of exposing immune cells comprising recombinant expression of the thrombopoietin receptor (hematopoietic growth factor receptor, c-MPL) to TPO and/or one or more agonists of c-MPL.

33. The method of claim 31, wherein the cancer comprises acute lymphoblastic leukemia, acute myelogenous leukemia, chronic lymphocytic leukemia, chronic myelogenous leukemia, acute monocytic leukemia, Hodgkin's lymphoma, non-Hodgkin's lymphoma, and/or solid tumors.

34. The method of claim 33, wherein the solid tumors comprise tumors of the brain, breast, bladder, bone, colon, rectum, cervix, endometrium, esophagus, eye, gallbladder, kidney, larynx and hypopharynx, liver, lung, nasopharynx, oropharynx, ovary, pancreas, penis, pituitary, prostate, skin, small intestine, stomach, testes, thymus, thyroid, uterus, vagina and/or vulva.

35. The method of claim 31, wherein the individual is provided one or more additional cancer therapies.

36. The method of claim 35, wherein the additional cancer therapies are chemotherapy, radiation, immunotherapy, surgery, or a combination thereof.
